(19) United States
(12) Patent Application Publication
(10) Pub. No.: US 2024/0151720 A1
(43) Pub. Date: May 9, 2024

(54) PROCESS FOR SELECTING CELL CLONES EXPRESSING A HETEROLOGOUS POLYPEPTIDE

(71) Applicant: Hoffmann-La Roche Inc., Little Falls, NJ (US)

(72) Inventors: Tobias Grosskopf, Muenchen (DE); Xenia Wezler, Muenchen (DE)

(73) Assignee: Hoffmann-La Roche Inc., Little Falls, NJ (US)

(21) Appl. No.: 18/377,668

(22) Filed: Oct. 6, 2023

Related U.S. Application Data
(63) Continuation of application No. PCT/EP2022/059173, filed on Apr. 7, 2022.

(30) Foreign Application Priority Data
Apr. 9, 2021 (EP) 21167535.0
Aug. 20, 2021 (EP) 21192298.4

(51) Int. Cl. G01N 33/569; G16B 40/20

(52) U.S. Cl. CPC G01N 33/56966; G16B 40/20

(57) ABSTRACT

Herein is reported a novel, high throughput suitable method for the characterization and identification as well as selection of recombinant cell clones expressing a bispecific antibody with high titers and low levels of antibody-related side products. The method according to the current invention uses the binding signals of an immunoassay-based screening of recombinant cell clone cultivation supernatants for isolated as well as simultaneous binding to the respective targets of the antibody. This data allows for the selection of high producer cell clones with desired product profile.

### CROSS-REFERENCE TO RELATED APPLICATIONS

This application is a continuation of International Application No. PCT/EP2022/059173 having an international filing date of Apr. 7, 2022, and which claims benefit of priority to European Patent Application No. 21167535.0, filed Apr. 9, 2012 and European Patent Application No. 21192298.4, filed Aug. 20, 2021, each of which are incorporated herein by reference in their entirety.

### FIELD OF THE INVENTION

The current invention is in the field of cell line generation. More precisely, herein is reported a method for selecting cell clones that express high amounts of correctly assembled and function heterologous polypeptide using ELISA-based data.

### BACKGROUND OF THE INVENTION

In recent years, recombinant polypeptide, such as antibodies and antibody-based molecules, have seen a wide application in the field of disease treatment.

Especially regarding antibodies, after the first wave of antibody-based drugs comprising the naturally occurring IgG format, the next generation of formats is now increasingly seeing molecules, which have substantial modifications and derivations from the classical IgG antibody format. The desire for targeting two or even more antigens sequentially or simultaneously brings up more and more complex antibody formats. With increasing format complexity the number of possible antibody-related side products, such as, e.g., chain mispairings, increases.

One step of a therapeutic antibody on its way to the market is the development of a stable producer cell line, which is characterized by high expression in combination with good quality of the recombinant protein, i.e. low antibody-related side product content. Generally, the search for novel drug molecules in the complex format antibody space is bottlenecked by the lack of high-throughput assays to assess product quality and function at low volumes during cell line development especially at single clone level.

Thus, to identify those rare cell clones, which are capable of producing correctly assembled complex antibodies, out of the large number of cell clones obtained after transfection, high-throughput methods with low required sample volume are needed. Such methods would provide the ability to shift the screening to earlier phases, whereby more monoclonal cell clones can be characterized and thereby evaluated. This increases the probability to identify an exceptional cell clone that can produce high quality grade complex format antibodies, thereby increasing the probability of technical success and at the same time minimizing the cost of goods.
Sawyer, W. S., et al. (Proc. Natl. Acad. Sci. USA 117 (2020) 9851-9856) reported a high-throughput antibody screening from complex matrices using intact protein electrospray mass spectrometry.
WO 2016/172485 reported multispecific antigen-binding proteins.

### SUMMARY OF THE INVENTION

Herein is reported a novel, high throughput suitable method for the characterization and identification as well as selection of recombinant cell clones expressing a complex antibody format with high titers and low levels of antibody-related side products. The method according to the current invention uses the binding signals of an immunoassay-based screening of recombinant cell clone cultivation supernatants for isolated as well as simultaneous binding to the respective targets of the antibody. This data allows for the selection of high producer cell clones with desired product profile at the 500 or more clone stage for the first time.

One aspect of the current invention is a method for identifying and/or selecting one or more recombinant cell clones from a multitude of (in one embodiment at least 500) recombinant cell clones all expressing the same (recombinant) (therapeutic) antibody, which is at least bispecific and which is (specifically) binding to a first and a second antigen, the method comprising the following steps:
cultivating each single deposited recombinant cell clone (for at least 5 days) to produce a cultivation supernatant;
determining
   i) the total protein (in certain embodiments, antibody) concentration in the supernatant (using an immunoassay);
   ii) the binding signal for each supernatant for binding to the first antigen of the (therapeutic) antibody in an immunoassay for at least 2, in one preferred embodiment (at least) 4, different concentrations of the supernatant;
   iii) the binding signal for each supernatant for binding to the second antigen of the (therapeutic) antibody in an immunoassay for the at least 2, in one preferred embodiment the (at least) 4, same concentrations of the supernatant as in ii);
   iv) the binding signal for each supernatant for simultaneous binding to the first and the second antigen of the (therapeutic) antibody in an (bridging) immunoassay for the at least 2, in one preferred embodiment the (at least) 4, same concentrations of the supernatant as in ii) and iii);
   v) the binding signal for the (isolated) (therapeutic) antibody (with a purity of at least 98% as determined by mass spectrometry) for isolated binding to the first antigen and the second antigen as well as simultaneous binding to both antigens with the same immunoassay as in ii), iii) and iv) each for at least 4, in one preferred embodiment at least 14, different concentrations of the (isolated) (therapeutic) antibody;
   vi) the binding signal for a first (the major/the main) (isolated) (therapeutic-)antibody-related side product, which can be also expressed/is also expressed by said cell clones, (with a purity of at least 70% as determined by mass spectrometry) for isolated binding to the first antigen and the second antigen as well as simultaneous binding to both antigens with the same immunoassay as in ii), iii) and iv) each for the at least 4, in one preferred embodiment the at least 14, same concentrations as in v) of the first (the major/the main) (therapeutic-)antibody-related side product;
   vii) the binding signal for a second (second major) (isolated) (therapeutic-)antibody-related side product, which can be also expressed/is also expressed by said cell clones, (with a purity of at least 70% as determined by mass spectrometry) for isolated binding to the first antigen and the second antigen as well as simultaneous binding to both antigens with the same immunoassay as in ii), iii) and iv) each for the at least 4, in one preferred embodiment the at least 14, same concentrations as in v) and vi) of the second (second major) (therapeutic-)antibody-related side product;
ranking the recombinant cell clones of the multitude of recombinant cell clones based on
   i), ii), iii), iv), and
   the median of (all) the ratios of the binding signal determined in iii) to the binding signal determined in ii) for the same concentration, whereby this median is further divided by (normalized to) the median of (all) the ratios of the binding signal determined in v) using the immunoassay of iii) to the binding signal determined in v) using the immunoassay of ii) for the same concentration, and
   the median of (all) the ratios of the binding signal determined in iv) to the binding signal determined in ii) for the same concentration, whereby this median is further divided by (normalized to) the median of (all) the ratios of the binding signal determined in v) using the immunoassay of iv) to the binding signal determined in v) using the immunoassay of ii) for the same concentration, and
   the median of (all) the ratios of the binding signal determined in iii) to the binding signal determined in ii) for the same concentration, whereby this median is further divided by (normalized to) the median of (all) the ratios of the binding signal determined in vi) using the immunoassay of iii) to the binding signal determined in vi) using the immunoassay of ii) for the same concentration, and
   the median of (all) the ratios of the binding signal determined in iv) to the binding signal determined in ii) for the same concentration, whereby this median is further divided by (normalized to) the median of (all) the ratios of the binding signal determined in vii) using the immunoassay of iii) to the binding signal determined in vii) using the immunoassay of ii) for the same concentration,
   (the single individual dimensions)
whereby the ranking is by the respective difference of the above values for each individual cell clone to the values obtained for the isolated (therapeutic) antibody,
whereby the recombinant cell clones with the smallest overall difference are selected.

In certain embodiments, the ranking is by the respective (absolute) difference (distance) of these values for each individual cell clone to the respective values obtained for the isolated (therapeutic) antibody used in v) in the assays of ii), iii) and iv) and the ratios derived therefrom.

In certain embodiments, the ranking is (a multidimensional ranking) taking into account the individual (absolute) distances for each individual value (dimension), i.e. based on the combination of the similarity (smallest (absolute) difference) with respect to the values for the (isolated) (therapeutic) antibody in combination to the dissimilarity (biggest (absolute) difference) with respect to the first (major) and the second (second major) isolated (therapeutic) antibody-related side product.

In certain embodiments, the selection is based on the smallest difference with respect to the concentration, the binding signals and the (therapeutic) antibody divided ratios in combination with the biggest difference with respect to the side product divided ratios.

In certain embodiments, the recombinant cell clones with the smallest overall, i.e. in all individual dimensions, (absolute) difference (distance) are identified/selected, i.e. those clones are identified/selected that have the smallest sum of all eight differences.

In certain embodiments, the one or more identified/selected cell clones express said recombinant (therapeutic) antibody with lower fraction or percentage of antibody-related side products as the average of the multitude of recombinant cell clones.

In certain embodiments, the difference (distance) is determined using an in silico method.

In certain embodiments, the difference (distance) is determined using machine learning.

In certain embodiments, the difference (distance) is determined using an extreme gradient boosting model.

In certain embodiments, the binding signals are the blank-corrected binding signals (raw binding signal reduced by the respective blank signal) In certain embodiments, in ii), iii) and iv) at least 4 concentrations are used. In certain embodiments, in ii), iii) and iv) 4 or 5 or 6 or 7 concentrations are used.

In certain embodiments, in v), vi) and vii) at least 14 concentrations are used. In certain embodiments, in v), vi) and vii) 14 or 15 or 16 or 17 or 18 concentrations are used.

In certain embodiments, the bispecific (therapeutic) antibody comprises a heterodimeric Fc-region, wherein one Fc-region polypeptide comprises the knob-mutation and the respective other Fc-region polypeptide comprises the hole-mutations; the first (major) bispecific (therapeutic) antibody-related side product is the hole-hole homodimer of said bispecific (therapeutic) antibody; the second (second major) bispecific (therapeutic) antibody-related side product is the knob-knob homodimer of said bispecific (therapeutic) antibody.

In certain embodiments, the immunoassay is an enzyme-linked immunosorbent assay.

In certain embodiments, the cell is a CHO cell.

In one preferred embodiment, the (therapeutic) antibody is a bispecific (therapeutic) antibody. In certain embodiments, the bispecific (therapeutic) antibody is heterodimeric with respect to its heavy chains, whereby one of the heavy chains comprises the hole-mutations and the respective other one comprises the knob-mutation. In certain embodiments, the first (the major/the main) (isolated) (therapeutic-)antibody-related side product comprises two heavy chains each with hole-mutations and the second (second major) (isolated) (therapeutic-)antibody-related side product comprises two heavy chains each with knob-mutation.

A further aspect of the current invention is the use of
i) the total antibody concentration determined in a single recombinant cell clone cultivation supernatant, wherein the recombinant cell clone has been transfected with nucleic acids encoding a bispecific (therapeutic) antibody,
ii) the binding signals for isolated binding of the supernatant to the first and second antigen of the bispecific (therapeutic) antibody,
iii) the binding signals for simultaneous binding of the supernatant to the first and second antigen of the bispecific (therapeutic) antibody for identifying recombinant cell clones expressing said bispecific (therapeutic) antibody with low bispecific antibody-related side products.

In certain embodiments, further
iv) the binding signals for isolated and simultaneous binding of the bispecific (therapeutic) antibody) to its first and second antigen,
v) the binding signals for isolated and simultaneous binding of a first (major) bispecific antibody-related side product to the first and second antigen of the bispecific (therapeutic) antibody,
vi) the binding signals for isolated and simultaneous binding of a second (second major) bispecific antibody-related side product to the first and second antigen of the bispecific (therapeutic) antibody
are used for identifying recombinant cell clones expressing said bispecific (therapeutic) antibody with low bispecific antibody-related side products.

In certain embodiments, further
the median of the individual ratios of the binding signal for each of the concentrations obtained in iii) to the respective binding signals for the same concentration obtained in ii) divided by the median of the individual ratios of the binding signal for each of the concentrations obtained in v) using the immunoassay of iii) to the respective binding signal for the same concentration obtained in v) using the immunoassay of ii),
the median of the individual ratios of the binding signal for each of the concentrations obtained in iv) to the respective binding signals for the same concentration obtained in ii) divided by the median of the individual ratios of the binding signal for each of the concentrations obtained in v) using the immunoassay of iv) to the respective binding signal for the same concentration obtained in v) using the immunoassay of ii),
the median of the individual ratios of the binding signal for each of the concentrations obtained in iii) to the respective binding signals for the same concentration obtained in ii) divided by the median of the individual ratios of the binding signal for each of the concentrations obtained in vi) using the immunoassay of iii) to the respective binding signal for the same concentration obtained in vi) using the immunoassay of ii), and
the median of the individual ratios of the binding signal for each of the concentrations obtained in iv) to the respective binding signals for the same concentration obtained in ii) divided by the median of the individual ratios of the binding signal for each of the concentrations obtained in vii) using the immunoassay of iv) to the respective binding signal for the same concentration obtained in vii) using the immunoassay of ii)
are used for identifying recombinant cell clones expressing said bispecific (therapeutic) antibody with low bispecific antibody-related side products,
wherein
iv) is the binding signals for isolated and simultaneous binding of the bispecific (therapeutic) antibody) to its first and second antigen,
v) is the binding signals for isolated and simultaneous binding of a first (major) bispecific antibody-related side product to the first and second antigen of the bispecific (therapeutic) antibody,
vi) is the binding signals for isolated and simultaneous binding of a second (second major) bispecific antibody-related side product to the first and second antigen of the bispecific (therapeutic) antibody.

A further aspect of the current invention is a computer program including computer-executable instructions for performing the method according to the invention when the program is executed on a computer or computer network.

A further aspect of the current invention is a computer program product having program code means, in order to perform the method according to the invention when the program is executed on a computer or computer network.

A further aspect of the invention is a data carrier having a data structure stored thereon, which, after loading into a computer or computer network, such as into a working memory or main memory of the computer or computer network, may execute the method according to the invention.

In addition to the various embodiments depicted and claimed, the disclosed subject matter is also directed to other embodiments having other combinations of the features disclosed and claimed herein. As such, the particular features presented herein, especially presented as aspects or embodiments, can be combined with each other in other manners within the scope of the disclosed subject matter such that the disclosed subject matter includes any suitable combination of the features disclosed herein. The description of specific embodiments of the disclosed subject matter has been presented for purposes of illustration and description. It is not intended to be exhaustive or to limit the disclosed subject matter to those embodiments disclosed.

An aspect as used herein relates to independent subject matter of the invention, an embodiment as used herein provides for a more detailed realization of one or more or all independent aspects.

### DETAILED DESCRIPTION OF THE INVENTION

The invention is based, at least in part, on the finding that using ELISA-based titer and binding characterization can be used to predict critical quality attributes of individual clones at the at least 500-clone stage during the cell line development phase of drug development.

The invention is further based, at least in part, on the finding that the combination of ELISA-based titer and binding characterization with an in silico data analysis can be used to predict critical quality attributes of individual clones at the at least 500-clone stage during the cell line development phase of drug development.

Thus, with the method according to the current invention the number of clones that can be analyzed is increased, the analysis times are reduced and at the same time reproducibility and ease of application is increased. Thereby it is possible for the first time to implement product quality screening early during cell line development, especially for complex antibody formats.

### General Definitions

Useful methods and techniques for carrying out the current invention are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and 11 (1985), Oxford University Press; Freshney, R. I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N.Y., VCH Publishers (1987); Celis, J., ed., Cell Biology, Second Edition, Academic Press (1998); Freshney, R. I., Culture of Animal Cells: A Manual of Basic Technique, second edition, Alan R. Liss, Inc., N.Y. (1987).

The use of recombinant DNA technology enables the generation of derivatives of a nucleic acid. Such derivatives can, for example, be modified in individual or several nucleotide positions by substitution, alteration, exchange, deletion or insertion. The modification or derivatization can, for example, be carried out by means of site directed mutagenesis. Such modifications can easily be carried out by a person skilled in the art (see e.g. Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) IRL Press, Oxford, England).

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

The term “about” denotes a range of +/−20% of the thereafter following numerical value. In certain embodiments, the term about denotes a range of +/−10% of the thereafter following numerical value. In certain embodiments, the term about denotes a range of +/−5% of the thereafter following numerical value.

The term “comprising” also encompasses the term “consisting of”.

### Cell-Specific Definitions

The term “cell clone” as used herein denotes a mammalian cell comprising an exogenous nucleotide sequence capable of expressing a polypeptide, i.e. a recombinant mammalian cell. Such recombinant mammalian cells are cells into which one or more exogenous nucleic acid(s) have been introduced, including the progeny of such cells. In certain embodiments, the cell clone is a mammalian cell comprising a nucleic acid encoding a heterologous polypeptide. Thus, the term “cell clone comprising a nucleic acid encoding a heterologous polypeptide” denotes recombinant mammalian cells comprising an exogenous nucleotide sequence integrated in the genome of the mammalian cell and capable of expressing the heterologous polypeptide. In certain embodiments, the cell clone is a mammalian cell comprising an exogenous nucleotide sequence integrated at a single site within a locus of the genome of the cell. In one preferred embodiment, the cell clone is a mammalian cell comprising an exogenous nucleotide sequence integrated at a single site within a locus of the genome of the cell, wherein the exogenous nucleotide sequence comprises a first and a second recombination recognition sequence flanking at least one first selection marker, and a third recombination recognition sequence located between the first and the second recombination recognition sequence, and all the recombination recognition sequences are different.

The term “recombinant cell” as used herein denotes a cell after genetic modification, such as, e.g., a cell expressing a heterologous polypeptide of interest and that can be used for the production of said heterologous polypeptide of interest at any scale. For example, “a cell clone” denotes a cell wherein the coding sequences for a heterologous polypeptide of interest have been introduced into the genome. For example, “a recombinant mammalian cell comprising an exogenous nucleotide sequence” that has been subjected to recombinase mediated cassette exchange (RMCE), whereby the coding sequences for a polypeptide of interest have been introduced into the genome of the host cell, is a specific “cell clone”.

A “cell clone” as used herein denotes a “transformed cell”. This includes the primary transformed cell as well as progeny derived therefrom without regard to the number of passages. Progeny may, e.g., not be completely identical in nucleic acid content to a parent cell, but may contain mutations. Mutant progeny that has the same function or biological activity as screened or selected for in the originally transformed cell are encompassed.

An “isolated cell clone” denotes a cell clone, which has been separated from a component of its natural environment.

An “isolated nucleic acid” denotes a nucleic acid molecule that has been separated from a component of its natural environment.

An “isolated polypeptide” or an “isolated antibody” or an “isolated side product” denotes a polypeptide molecule or an antibody molecule, respectively, that has been separated from a component of its natural environment. In certain embodiments, an isolated polypeptide or an isolated antibody or an isolated side product is purified to greater than 70% purity as determined by, for example, mass spectrometry. In certain embodiments, an isolated polypeptide or an isolated antibody or an isolated side product is purified to greater than 95% or 98% purity as determined by, for example, mass spectrometry. For review of methods for assessment of e.g. antibody purity, see, e.g., Flatman, S. et al., J. Chrom. B 848 (2007) 79-87.

### Assays

The principles of different immunoassays are described, for example, by Hage, D. S. (Anal. Chem. 71 (1999) 294R-304R). Lu, B., et al. (Analyst 121 (1996) 29R-32R) report the orientated immobilization of antibodies for the use in immunoassays. Avidin-biotin-mediated immunoassays are reported, for example, by Wilchek, M., and Bayer, E. A., in Methods Enzymol. 184 (1990) 467-469.

The term “immunoassay” denotes any technique that utilizes specifically binding molecules, such as antibodies, to capture and/or detect a specific target for qualitative or quantitative analysis. In general, an immunoassay is characterized by the following steps: 1) immobilization or capture of the analyte and 2) detection and measuring the analyte. The analyte can be captured, i.e. bound, on any solid surface, such as e.g. a membrane, plastic plate, or some other solid surface. A specific form of an immunoassay is an ELISA (enzyme-linked immunosorbent assay).

Generally, immunoassays can be performed in three different formats. One is with direct detection, one with indirect detection, or by a sandwich assay.

The direct detection immunoassay uses a detection (or tracer) antibody that can be measured directly. An enzyme or other molecule allows for the generation of a signal that will produce a color, fluorescence, or luminescence that allow the signal to be visualized or measured (radioisotopes can also be used, although it is not commonly used today).

In an indirect assay, a primary antibody that binds to the analyte is used to provide a defined target for a secondary antibody (tracer antibody) that specifically binds to the target provided by the primary antibody (referred to as detector or tracer antibody). The secondary antibody generates the measurable signal.

The sandwich assay makes use of two antibodies, a capture and a trace (detector) antibody. The capture antibody is used to bind (immobilize) analyte from solution or bind to it in solution. This allows the analyte to be specifically removed from the sample. The tracer (detector) antibody is used in a second step to generate a signal (either directly or indirectly as described above). The sandwich format requires two antibodies each with a distinct epitope on the target molecule. In addition, they must not interfere with one another, as both antibodies must be bound to the target at the same time.

Monoclonal antibodies and their constant domains contain a number of reactive amino acid side chains for conjugating to a member of a binding pair, such as a polypeptide/protein, a polymer (e.g. PEG, cellulose or polystyrol), or an enzyme. Chemical reactive groups of amino acids are, for example, amino groups (lysins, alpha-amino groups), thiol groups (cystins, cysteines, and methionins), carboxylic acid groups (aspartic acids, glutamic acids), and sugar-alcoholic groups. Such methods are e.g. described by Aslam M., and Dent, A., in “Bioconjugation”, MacMillan Ref. Ltd. 1999, pages 50-100.

One of the most common reactive groups of antibodies is the aliphatic ε-amine of the amino acid lysine. In general, nearly all antibodies contain abundant lysine. Lysine amines are reasonably good nucleophiles above pH 8.0 (pKa=9.18) and therefore react easily and cleanly with a variety of reagents to form stable bonds. Amine-reactive reagents react primarily with lysins and the α-amino groups of proteins. Reactive esters, particularly N-hydroxy-succinimide (NHS) esters, are among the most commonly employed reagents for modification of amine groups. The optimum pH for reaction in an aqueous environment is pH 8.0 to 9.0. Isothiocyanates are amine-modification reagents and form thiourea bonds with proteins. They react with protein amines in aqueous solution (optimally at pH 9.0 to 9.5). Aldehydes react under mild aqueous conditions with aliphatic and aromatic amines, hydrazines, and hydrazides to form an imine intermediate (Schiff's base). A Schiff's base can be selectively reduced with mild or strong reducing agents (such as sodium borohydride or sodium cyanoborohydride) to derive a stable alkyl amine bond. Other reagents that have been used to modify amines are acid anhydrides. For example, diethylenetriaminepentaacetic anhydride (DTPA) is a bifunctional chelating agent that contains two amine-reactive anhydride groups. It can react with N-terminal and ε-amine groups of amino acids to form amide linkages. The anhydride rings open to create multivalent, metal-chelating arms able to bind tightly to metals in a coordination complex.

Another common reactive group in antibodies is the thiol residue from the sulfur-containing amino acid cystine and its reduction product cysteine (or half cystine). Cysteine contains a free thiol group, which is more nucleophilic than amines and is generally the most reactive functional group in a protein. Thiols are generally reactive at neutral pH, and therefore can be coupled to other molecules selectively in the presence of amines. Since free sulfhydryl groups are relatively reactive, proteins with these groups often exist with them in their oxidized form as disulfide groups or disulfide bonds. In such proteins, reduction of the disulfide bonds with a reagent such as dithiotreitol (DTT) is required to generate the reactive free thiol. Thiol-reactive reagents are those that will couple to thiol groups on polypeptides, forming thioether-coupled products. These reagents react rapidly at slight acidic to neutral pH and therefore can be reacted selectively in the presence of amine groups. The literature reports the use of several thiolating crosslinking reagents such as Traut's reagent (2-iminothiolane), succinimidyl (acetylthio) acetate (SATA), and sulfosuccinimidyl 6-[3-(2-pyridyldithio) propionamido] hexanoate (Sulfo-LC-SPDP) to provide efficient ways of introducing multiple sulfhydryl groups via reactive amino groups. Haloacetyl derivatives, e.g. iodoacetamides, form thioether bonds and are also reagents for thiol modification. Further useful reagents are maleimides. The reaction of maleimides with thiol-reactive reagents is essentially the same as with iodoacetamides. Maleimides react rapidly at slight acidic to neutral pH.

Another common reactive group in antibodies are carboxylic acids. Antibodies contain carboxylic acid groups at the C-terminal position and within the side chains of aspartic acid and glutamic acid. The relatively low reactivity of carboxylic acids in water usually makes it difficult to use these groups to selectively modify polypeptides and antibodies. When this is done, the carboxylic acid group is usually converted to a reactive ester by the use of a water-soluble carbodiimide and reacted with a nucleophilic reagent such as an amine, hydrazide, or hydrazine. The amine-containing reagent should be weakly basic in order to react selectively with the activated carboxylic acid in the presence of the more highly basic F-amines of lysine to form a stable amide bond. Protein crosslinking can occur when the pH is raised above 8.0.

Sodium periodate can be used to oxidize the alcohol part of a sugar within a carbohydrate moiety attached to an antibody to an aldehyde. Each aldehyde group can be reacted with an amine, hydrazide, or hydrazine as described for carboxylic acids. Since the carbohydrate moiety is predominantly found on the crystallizable fragment region (Fc-region) of an antibody, conjugation can be achieved through site-directed modification of the carbohydrate away from the antigen-binding site. A Schiff's base intermediate is formed, which can be reduced to an alkyl amine through the reduction of the intermediate with sodium cyanoborohydride (mild and selective) or sodium borohydride (strong) water-soluble reducing agents.

The conjugation of a tracer and/or capture and/or detection antibody to its conjugation partner can be performed by different methods, such as chemical binding, or binding via a binding pair. The term “conjugation partner” as used herein denotes e.g. solid supports, polypeptides, detectable labels, members of specific binding pairs. In certain embodiments, the conjugation of the capture and/or tracer and/or detection antibody to its conjugation partner is performed by chemically binding via N-terminal and/or ε-amino groups (lysine), ε-amino groups of different lysins, carboxy-, sulfhydryl-, hydroxyl-, and/or phenolic functional groups of the amino acid backbone of the antibody, and/or sugar alcohol groups of the carbohydrate structure of the antibody. In certain embodiments, the capture antibody is conjugated to its conjugation partner via a binding pair. In one preferred embodiment, the capture antibody is conjugated to biotin and immobilization to a solid support is performed via solid support immobilized avidin or streptavidin. In certain embodiments, the tracer antibody is conjugated to its conjugation partner via a binding pair. In one preferred embodiment, the tracer antibody is conjugated to digoxygenin by a covalent bond as detectable label.

The term “solid phase” denotes a non-fluid substance, and includes particles (including microparticles and beads) made from materials such as polymer, metal (paramagnetic, ferromagnetic particles), glass, and ceramic; gel substances such as silica, alumina, and polymer gels; capillaries, which may be made of polymer, metal, glass, and/or ceramic; zeolites and other porous substances; electrodes; microtiter plates; solid strips; and cuvettes, tubes or other spectrometer sample containers. A solid phase component is distinguished from inert solid surfaces in that a “solid phase” contains at least one moiety on its surface, which is intended to interact with a substance in a sample. A solid phase may be a stationary component, such as a tube, strip, cuvette or microtiter plate, or may be non-stationary components, such as beads and microparticles. A variety of microparticles that allow both non-covalent or covalent attachment of proteins and other substances may be used. Such particles include polymer particles such as polystyrene and poly (methyl methacrylate); gold particles such as gold nanoparticles and gold colloids; and ceramic particles such as silica, glass, and metal oxide particles. See for example Martin, C. R., et al., Analytical Chemistry-News & Features, 70 (1998) 322A-327A, or Butler, J. E., Methods 22 (2000) 4-23.

Chromogens (fluorescent or luminescent groups and dyes), enzymes, NMR-active groups or metal particles, haptens, e.g. digoxygenin, are examples of “detectable labels”. The detectable label can also be a photoactivatable crosslinking group, e.g. an azido or an azirine group. Metal chelates, which can be detected by electrochemiluminescense, are also signal-emitting groups, with particular preference being given to ruthenium chelates, e.g. a ruthenium (bispyridyl)₃²⁺ chelate. Suitable ruthenium labeling groups are described, for example, in EP 0 580 979, WO 90/05301, WO 90/11511, and WO 92/14138. For direct detection, the labeling group can be selected from any known detectable marker groups, such as dyes, luminescent labeling groups such as chemiluminescent groups, e.g. acridinium esters or dioxetanes, or fluorescent dyes, e.g. fluorescein, coumarin, rhodamine, oxazine, resorufin, cyanine and derivatives thereof. Other examples of labeling groups are luminescent metal complexes, such as ruthenium or europium complexes, enzymes, e.g. as used for ELISA or for CEDIA (Cloned Enzyme Donor Immunoassay, e.g. EP-A-0 061 888), and radioisotopes.

Indirect detection systems comprise, for example, that the detection reagent, e.g., the detection antibody is labeled with a first partner of a binding pair. Examples of suitable binding pairs are antigen/antibody, biotin or biotin analogues such as aminobiotin, iminobiotin or desthiobiotin/avidin or Streptavidin, sugar/lectin, nucleic acid or nucleic acid analogue/complementary nucleic acid, and receptor/ligand, e.g., steroid hormone receptor/steroid hormone. In one preferred embodiment, the first binding pair members comprise hapten, antigen and hormone. In certain embodiments, the hapten is selected from the group consisting of digoxin, digoxygenin and biotin and analogues thereof. The second partner of such binding pair, e.g. an antibody, Streptavidin, etc., usually is labeled to allow for direct detection, e.g., by the labels as mentioned above.

### Antibodies

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, MD (1991).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991) and is referred to as “numbering according to Kabat” herein. Specifically, the Kabat numbering system (see pages 647-660) of Kabat, et al., Sequences of Proteins of Immunological Interest, 5th ed., Public Health Service, National Institutes of Health, Bethesda, MD (1991) is used for the light chain constant domain CL of kappa and lambda isotype, and the Kabat EU index numbering system (see pages 661-723) of Kabat, et al., Sequences of Proteins of Immunological Interest, 5th ed., Public Health Service, National Institutes of Health, Bethesda, MD (1991) is used for the constant heavy chain domains (CH1, hinge, CH2 and CH3, which is herein further clarified by referring to “numbering according to Kabat EU index” in this case).

The term “antibody” herein is used in the broadest sense and encompasses various antibody structures, including but not limited to full length antibodies, monoclonal antibodies, multispecific antibodies (e.g., bispecific antibodies), and antibody-antibody fragment-fusions as well as combinations thereof.

The term “native antibody” denotes naturally occurring immunoglobulin molecules with varying structures. For example, native IgG antibodies are heterotetrameric glycoproteins of about 150,000 Daltons, composed of two identical light chains and two identical heavy chains that are disulfide-bonded. From N- to C-terminus, each heavy chain has a heavy chain variable region (VH) followed by three heavy chain constant domains (CH1, CH2, and CH3), whereby between the first and the second heavy chain constant domain a hinge region is located. Similarly, from N- to C-terminus, each light chain has a light chain variable region (VL) followed by a light chain constant domain (CL). The light chain of an antibody may be assigned to one of two types, called kappa (κ) and lambda (λ), based on the amino acid sequence of its constant domain.

The term “full length antibody” denotes an antibody having a structure substantially similar to that of a native antibody. A full length antibody comprises two full length antibody light chains each comprising in N- to C-terminal direction a light chain variable region and a light chain constant domain, as well as two full length antibody heavy chains each comprising in N- to C-terminal direction a heavy chain variable region, a first heavy chain constant domain, a hinge region, a second heavy chain constant domain and a third heavy chain constant domain. In contrast to a native antibody, a full length antibody may comprise further immunoglobulin domains, such as e.g. one or more additional scFvs, or heavy or light chain Fab fragments, or scFabs conjugated to one or more of the termini of the different chains of the full length antibody, but only a single fragment to each terminus. These conjugates are also encompassed by the term full-length antibody.

The term “antibody binding site” denotes a pair of a heavy chain variable domain and a light chain variable domain. To ensure proper binding to the antigen these variable domains are cognate variable domains, i.e. belong together. An antibody the binding site comprises at least three HVRs (e.g. in case of a VHH) or three-six HVRs (e.g. in case of a naturally occurring, i.e. conventional, antibody with a VH/VL pair). Generally, the amino acid residues of an antibody that are responsible for antigen binding are forming the binding site. These residues are normally contained in a pair of an antibody heavy chain variable domain and a corresponding antibody light chain variable domain. The antigen-binding site of an antibody comprises amino acid residues from the “hypervariable regions” or “HVRs”. “Framework” or “FR” regions are those variable domain regions other than the hypervariable region residues as herein defined. Therefore, the light and heavy chain variable domains of an antibody comprise from N- to C-terminus the regions FR1, HVR1, FR2, HVR2, FR3, HVR3 and FR4. Especially, the HVR3 region of the heavy chain variable domain is the region, which contributes most to antigen binding and defines the binding specificity of an antibody. A “functional binding site” is capable of binding to its target. The term “binding to” denotes the binding of a binding site to its target in an in vitro assay, in certain embodiments, in a binding assay. Such binding assay can be any assay as long the binding event can be detected. “Binding” can be determined using, for example, an ELISA assay.

The term “hypervariable region” or “HVR”, as used herein, refers to each of the regions of an antibody variable domain comprising the amino acid residue stretches which are hypervariable in sequence (“complementarity determining regions” or “CDRs”) and/or form structurally defined loops (“hypervariable loops”), and/or contain the antigen-contacting residues (“antigen contacts”). Generally, antibodies comprise six HVRs; three in the heavy chain variable domain VH (H1, H2, H3), and three in the light chain variable domain VL (L1, L2, L3).

### HVRs Include

(a) hypervariable loops occurring at amino acid residues 26-32 (L1), 50-52 (L2), 91-96 (L3), 26-32 (H1), 53-55 (H2), and 96-101 (H3) (Chothia, C. and Lesk, A. M., J. Mol. Biol. 196 (1987) 901-917);
(b) CDRs occurring at amino acid residues 24-34 (L1), 50-56 (L2), 89-97 (L3), 31-35b (H1), 50-65 (H2), and 95-102 (H3) (Kabat, E. A. et al., Sequences of Proteins of Immunological Interest, 5th ed. Public Health Service, National Institutes of Health, Bethesda, MD (1991), NIH Publication 91-3242.);
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

The “class” of an antibody refers to the type of constant domains or constant region, preferably the Fc-region, possessed by its heavy chains. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called u, 6, F, y, and p, respectively.

The term “heavy chain constant region” denotes the region of an immunoglobulin heavy chain that contains the constant domains, i.e. the CH1 domain, the hinge region, the CH2 domain and the CH3 domain. In one embodiment, a human IgG constant region extends from Ala118 to the carboxyl-terminus of the heavy chain (numbering according to Kabat EU index). However, the C-terminal lysine (Lys447) of the constant region may or may not be present (numbering according to Kabat EU index). The term “constant region” denotes a dimer comprising two heavy chain constant regions, which can be covalently linked to each other via the hinge region cysteine residues forming inter-chain disulfide bonds.

The term “heavy chain Fc-region” denotes the C-terminal region of an immunoglobulin heavy chain that contains at least a part of the hinge region (middle and lower hinge region), the CH2 domain and the CH3 domain. In one embodiment, a human IgG heavy chain Fc-region extends from Asp221, or from Cys226, or from Pro230, to the carboxyl-terminus of the heavy chain (numbering according to Kabat EU index). Thus, an Fc-region is smaller than a constant region but in the C-terminal part identical thereto. However, the C-terminal lysine (Lys447) of the heavy chain Fc-region may or may not be present (numbering according to Kabat EU index). The term “Fc-region” denotes a dimer comprising two heavy chain Fc-regions, which can be covalently linked to each other via the hinge region cysteine residues forming inter-chain disulfide bonds.

The term “valent” as used within the current application denotes the presence of a specified number of binding sites in an antibody. As such, the terms “bivalent”, “tetravalent”, and “hexavalent” denote the presence of two binding site, four binding sites, and six binding sites, respectively, in an antibody.

A “monospecific antibody” denotes an antibody that has a single binding specificity, i.e. specifically binds to one antigen. Monospecific antibodies can be prepared as full-length antibodies or antibody fragments (e.g. F(ab′)₂) or combinations thereof (e.g. full length antibody plus additional scFv or Fab fragments). A monospecific antibody does not need to be monovalent, i.e. a monospecific antibody may comprise more than one binding site specifically binding to the one antigen. A native antibody, for example, is monospecific but bivalent.

A “multispecific antibody” denotes an antibody that has binding specificities for at least two different epitopes on the same antigen or two different antigens. Multispecific antibodies can be prepared as full-length antibodies or antibody fragments (e.g. Fab bispecific antibodies) or combinations thereof (antibody-antibody fragment-fusions, e.g. a full-length antibody conjugated to an additional scFv or Fab fragments). A multispecific antibody is at least bivalent, i.e. comprises two antigen binding sites. In addition, a multispecific antibody is at least bispecific. Thus, a bivalent, bispecific antibody is the simplest form of a multispecific antibody. Engineered antibodies with two, three or more (e.g. four) functional antigen binding sites have also been reported (see, e.g., US 2002/0004587).

In certain embodiments, the cell clone produces/the cell clones produce a multispecific antibody. In certain embodiments, one of the binding specificities is for a first antigen and the other is for a different second antigen. In certain embodiments, the multispecific antibody binds to two different epitopes of the same antigen. In certain embodiments, the second epitope on the same antigen is a non-overlapping epitope. In certain embodiments, the antibody is a bispecific antibody. In one preferred embodiment, the bispecific antibody is a trivalent, bispecific antibody or a bivalent, bispecific antibody.

Techniques for making multispecific antibodies include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A., et al., EMBO J. 10 (1991) 3655-3659), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004); cross-linking two or more antibodies or fragments (see, e.g., U.S. Pat. No. 4,676,980, and Brennan, M., et al., Science 229 (1985) 81-83); using leucine zippers to produce bi-specific antibodies (see, e.g., Kostelny, S. A., et al., J. Immunol. 148 (1992) 1547-1553); using the common light chain technology for circumventing the light chain mis-pairing problem (see, e.g., WO 98/50431); using specific technology for making bispecific antibody fragments (see, e.g., Holliger, P., et al., Proc. Natl. Acad. Sci. USA 90 (1993) 6444-6448); and preparing trispecific antibodies as described, e.g., in Tutt, A., et al., J. Immunol. 147 (1991) 60-69).

Engineered antibodies with three or more antigen binding sites, including for example, “Octopus antibodies”, or DVD-Ig are also included herein (see, e.g., WO 2001/77342 and WO 2008/024715). Other examples of multispecific antibodies with three or more antigen binding sites can be found in WO 2010/115589, WO 2010/112193, WO 2010/136172, WO 2010/145792, and WO 2013/026831. The bispecific antibody or antigen binding fragment thereof also includes a “Dual Acting Fab” or “DAF” (see, e.g., US 2008/0069820 and WO 2015/095539).

Multi-specific antibodies may also be provided in an asymmetric form with a domain crossover in one or more binding arms of the same antigen specificity, i.e. by exchanging the VH/VL domains (see, e.g., WO 2009/080252 and WO 2015/150447), the CH1/CL domains (see, e.g., WO 2009/080253) or the complete Fab arms (see e.g., WO 2009/080251, WO 2016/016299, also see Schaefer et al., Proc. Natl. Acad. Sci. USA 108 (2011) 1187-1191, and Klein at al., MAbs 8 (2016) 1010-1020).

In one preferred embodiment, the multispecific antibody comprises a Fab fragment, in which either the variable regions or the constant regions of the heavy and light chain are exchanged, i.e. wherein in one chain a heavy chain VH variable domain is either directly of via a peptidic linker conjugated to a light chain CL constant domain, and in the respective other chain a light chain VL variable domain is either directly of via a peptidic linker conjugated to a heavy chain CH1 constant domain.

Thus, a domain exchanged Fab fragment comprises a polypeptide chain composed of the light chain variable region (VL) and the heavy chain constant region 1 (CH1), and a polypeptide chain composed of the heavy chain variable region (VH) and the light chain constant region (CL).

Asymmetrical Fab arms can also be engineered by introducing charged or non-charged amino acid mutations into domain interfaces to direct correct Fab pairing. See e.g., WO 2016/172485.

The antibody or fragment can also be a multispecific antibody as described in WO 2009/080254, WO 2010/112193, WO 2010/115589, WO 2010/136172, WO 2010/145792, or WO 2010/145793.

The antibody or fragment thereof may also be a multispecific antibody as disclosed in WO 2012/163520.

Various further molecular formats for multispecific antibodies are known in the art and are included herein (see e.g., Spiess et al., Mol. Immunol. 67 (2015) 95-106).

Bispecific antibodies are generally antibody molecules that specifically bind to two different, non-overlapping epitopes on the same antigen or to two epitopes on different antigens.

In certain embodiments, the bispecific antibody is selected from the group of bispecific antibodies consisting of
a domain exchanged 1+1 bispecific antibody (CrossMab)
   (a bispecific, full-length IgG antibody comprising a pair of a first light chain and a first heavy chain comprising a first Fab fragment and a pair of a second light chain and a second heavy chain comprising a second Fab fragment,
   wherein in the first Fab fragment
   a) only the CH1 and CL domains are replaced by each other (i.e. the light chain of the first Fab fragment comprises a VL and a CH1 domain and the heavy chain of the first Fab fragment comprises a VH and a CL domain);
   b) only the VH and VL domains are replaced by each other (i.e. the light chain of the first Fab fragment comprises a VH and a CL domain and the heavy chain of the first Fab fragment comprises a VL and a CH1 domain); or
   c) the CH1 and CL domains and the VH and VL domains are replaced by each other (i.e. the light chain of the first Fab fragment comprises a VH and a CH1 domain and the heavy chain of the first Fab fragment comprises a VL and a CL domain);
   wherein the second Fab fragment comprises a light chain comprising a VL and a CL domain, and a heavy chain comprising a VH and a CH1 domain;
   wherein the first heavy chain and the second heavy chain both comprise a CH3 domain, wherein both CH3 domains are engineered in a complementary manner by respective amino acid substitutions, in order to support heterodimerization of the first heavy chain and the second heavy chain, (in one preferred embodiment, one CH3 domain comprises the knob-mutation and the respective other CH3 domain comprises the hole-mutations);
C-terminal Fab domain fused 2+1 bispecific antibody (BS)
   (a bispecific, full length IgG antibody comprising
   a) one full length antibody comprising two pairs each of a full length antibody light chain and a full length antibody heavy chain, wherein the binding sites formed by each of the pairs of the full length heavy chain and the full length light chain specifically bind to a first antigen, and
   b) one additional Fab fragment, wherein the additional Fab fragment is fused to the C-terminus of one heavy chain of the full length antibody, wherein the binding site of the additional Fab fragment specifically binds to a second antigen,
   wherein the additional Fab fragment specifically binding to the second antigen comprises a domain crossover such that a) the light chain variable domain (VL) and the heavy chain variable domain (VH) are replaced by each other, or b) the light chain constant domain (CL) and the heavy chain constant domain (CH1) are replaced by each other);
a bispecific, one-armed single chain antibody
   (a bispecific, one-armed single chain antibody comprising a first binding site that specifically binds to a first epitope or antigen and a second binding site that specifically binds to a second epitope or antigen, whereby the individual chains are as follows
      a light chain (comprising variable light chain domain and light chain constant domain);
      a combined light/heavy chain (comprising in N- to C-terminal order a variable light chain domain, a light chain constant domain, peptidic linker, variable heavy chain domain, a CH1 domain, a hinge region, a CH2 domain and a CH3 with knob- or hole-mutation)
      a heavy chain (comprising in N- to C-terminal order a variable heavy chain domain, a CH1 domain, a hinge region, a CH2 domain, a CH3 domain with hole- or knob-mutation));
a bispecific, two-armed single chain antibody
   (a bispecific, two-armed single chain antibody comprising a first binding site that specifically binds to a first epitope or antigen and a second binding site that specifically binds to a second epitope or antigen, whereby the individual chains are as follows
      a combined light/heavy chain 1 (comprising in N- to C-terminal order a variable light chain domain 1, a light chain constant domain, a peptidic linker, a variable heavy chain domain 1, a CH1 domain, a hinge region, a CH2 domain, a CH3 domain with knob- or hole-mutation);
      combined light/heavy chain 2 (comprising in N- to C-terminal order a variable light chain domain 2, a light chain constant domain, a peptidic linker, a variable heavy chain domain 2, a CH1 domain, a hinge region, a CH2 domain, a CH3 domain with hole- or knob-mutation));
a common light chain bispecific antibody
   (a common light chain bispecific antibody comprising a first binding site that specifically binds to a first epitope or antigen and a second binding site that specifically binds to a second epitope or antigen, whereby the individual chains are as follows
      a light chain (comprising in N- to C-terminal order a variable light chain domain and a light chain constant domain);
      a heavy chain 1 (comprising in N- to C-terminal order a variable heavy chain domain 1, a CH1 domain, a hinge region, a CH2 domain, a CH3 domain with hole- or knob-mutation);
      heavy chain 2 (comprising in N- to C-terminal order a variable heavy chain domain 2, a CH1 domain, a hinge region, a CH2 domain, a CH3 domain with knob- or hole-mutation));
N-terminal Fab-domain inserted 2+1 bispecific antibody (TCB)
   (a bispecific, full-length antibody with additional heavy chain N-terminal binding site with domain exchange comprising
      a first and a second Fab fragment, wherein each binding site of the first and the second Fab fragment specifically bind to a first antigen,
      a third Fab fragment, wherein the binding site of the third Fab fragment specifically binds to a second antigen, and wherein the third Fab fragment comprises a domain crossover such that the variable light chain domain (VL) and the variable heavy chain domain (VH) are replaced by each other, and
      an Fc-region comprising a first Fc-region polypeptide and a second Fc-region polypeptide,
      wherein the first and the second Fab fragment each comprise a heavy chain fragment and a full-length light chain,
      wherein the C-terminus of the heavy chain fragment of the first Fab fragment is fused to the N-terminus of the first Fc-region polypeptide,
      wherein the C-terminus of the heavy chain fragment of the second Fab fragment is fused to the N-terminus of the variable light chain domain of the third Fab fragment and the C-terminus of the CH1 domain of the third Fab fragment is fused to the N-terminus of the second Fc-region polypeptide);
an antibody-multimer-fusion
   (fusion polypeptide comprising
      (a) an antibody heavy chain and an antibody light chain, and
      (b) a first fusion polypeptide comprising in N- to C-terminal direction a first part of a non-antibody multimeric polypeptide, an antibody heavy chain CH1 domain or an antibody light chain constant domain, an antibody hinge region, an antibody heavy chain CH2 domain and an antibody heavy chain CH3 domain, and a second fusion polypeptide comprising in N- to C-terminal direction the second part of the non-antibody multimeric polypeptide and an antibody light chain constant domain if the first polypeptide comprises an antibody heavy chain CH1 domain or an antibody heavy chain CH1 domain if the first polypeptide comprises an antibody light chain constant domain,
      wherein
         (i) the antibody heavy chain of (a) and the first fusion polypeptide of (b), (ii) the antibody heavy chain of (a) and the antibody light chain of (a), and (iii) the first fusion polypeptide of (b) and the second fusion polypeptide of (b) are each independently of each other covalently linked to each other by at least one disulfide bond,
      wherein
         the variable domains of the antibody heavy chain and the antibody light chain form a binding site specifically binding to an antigen).

The CH3 domains in the heavy chains of an antibody can be altered by the “knob-into-holes” technology, which is described in detail with several examples in e.g. WO 96/027011, Ridgway, J. B., et al., Protein Eng. 9 (1996) 617-621; and Merchant, A. M., et al., Nat. Biotechnol. 16 (1998) 677-681. In this method, the interaction surfaces of the two CH3 domains are altered to increase the heterodimerization of these two CH3 domains and thereby of the polypeptide comprising them. Each of the two CH3 domains (of the two heavy chains) can be the “knob”, while the respective other is the “hole”. The introduction of a disulfide bridge further stabilizes the heterodimers (Merchant, A. M., et al., Nature Biotech. 16 (1998) 677-681; Atwell, S., et al., J. Mol. Biol. 270 (1997) 26-35) and increases the yield.

The mutation T366W in the CH3 domain (of an antibody heavy chain) is denoted as “knob-mutation” and the mutations T366S, L368A, Y407V in the CH3 domain (of an antibody heavy chain) are denoted as “hole-mutations” (numbering according to Kabat EU index). An additional inter-chain disulfide bridge between the CH3 domains can also be used (Merchant, A. M., et al., Nature Biotech. 16 (1998) 677-681) e.g. by introducing a S354C mutation into the CH3 domain of the heavy chain with the “knob-mutation” (denotes as “knob-cys-mutations”) and by introducing a Y349C mutation into the CH3 domain of the heavy chain with the “hole-mutations” (denotes as “hole-cys-mutations”) (numbering according to Kabat EU index).

The term “domain crossover” as used herein denotes that in a pair of an antibody heavy chain VH-CH1 fragment and its corresponding cognate antibody light chain, i.e. in an antibody Fab (fragment antigen binding), the domain sequence deviates from the sequence in a native antibody in that at least one heavy chain domain is substituted by its corresponding light chain domain and vice versa. There are three general types of domain crossovers, (i) the crossover of the CH1 and the CL domains, which leads by the domain crossover in the light chain to a VL-CH1 domain sequence and by the domain crossover in the heavy chain fragment to a VH-CL domain sequence (or a full length antibody heavy chain with a VH-CL-hinge-CH2-CH3 domain sequence), (ii) the domain crossover of the VH and the VL domains, which leads by the domain crossover in the light chain to a VH-CL domain sequence and by the domain crossover in the heavy chain fragment to a VL-CH1 domain sequence, and (iii) the domain crossover of the complete light chain (VL-CL) and the complete VH-CH1 heavy chain fragment (“Fab crossover”), which leads to by domain crossover to a light chain with a VH-CH1 domain sequence and by domain crossover to a heavy chain fragment with a VL-CL domain sequence (all aforementioned domain sequences are indicated in N-terminal to C-terminal direction).

As used herein the term “replaced by each other” with respect to corresponding heavy and light chain domains refers to the aforementioned domain crossovers. As such, when CH1 and CL domains are “replaced by each other” it is referred to the domain crossover mentioned under item (i) and the resulting heavy and light chain domain sequence. Accordingly, when VH and VL are “replaced by each other”, it is referred to the domain crossover mentioned under item (ii); and when the CH1 and CL domains are “replaced by each other” and the VH and VL domains are “replaced by each other” it is referred to the domain crossover mentioned under item (iii). Bispecific antibodies including domain crossovers are reported, e.g. in WO 2009/080251, WO 2009/080252, WO 2009/080253, WO 2009/080254 and Schaefer, W., et al, Proc. Natl. Acad. Sci. USA 108 (2011) 11187-11192. Such antibodies are generally termed CrossMab.

Multispecific antibodies also comprise, in certain embodiments, at least one Fab fragment including either a domain crossover of the CH1 and the CL domains as mentioned under item (i) above, or a domain crossover of the VH and the VL domains as mentioned under item (ii) above, or a domain crossover of the VH-CH1 and the VL-VL domains as mentioned under item (iii) above. In case of multispecific antibodies with domain crossover, the Fabs specifically binding to the same antigen(s) are constructed, in certain embodiments, to be of the same domain sequence. Hence, in case more than one Fab with a domain crossover is contained in the multispecific antibody, said Fab(s) specifically bind to the same antigen.

The term “recombinant antibody”, as used herein, denotes all antibodies (chimeric, humanized and human) that are prepared, expressed, created or isolated by recombinant means, such as recombinant cells. This includes antibodies isolated from recombinant cells such as NS0, HEK, BHK, amniocyte or CHO cells.

As used herein, the term “antibody fragment” refers to a molecule other than an intact antibody that comprises a portion of an intact antibody that binds the antigen to which the intact antibody binds, i.e. it is a functional fragment. Examples of antibody fragments include but are not limited to Fv; Fab; Fab′; Fab′-SH; F(ab′)2; bispecific Fab; diabodies; linear antibodies; single-chain antibody molecules (e.g., scFv or scFab).

The term “correctly assembled antibody” and grammatical equivalents thereof refers to an antibody assembled from cognate pairs of heavy and light chains. That is, a correctly assembled antibody comprises for each chain the respective pairing partner. For example, each heavy chain is paired with its cognate light chain, each heavy chain Fab fragment is paired with its cognate light chain and/or each scFab fragment is paired with itself and not a different light chain of heavy chain Fab fragment.

The term “antibody-related side product” refers to molecules obtained during the expression or concomitantly with the expression of a recombinant antibody that are not the correctly assembled antibody but comprise fewer or more antibody chains (polypeptides) than the correctly assembled antibody. An antibody-related side product encompasses, for example, antibody molecules wherein one or more chains, such as light chains, are missing, or wherein one or more heavy chains or heavy chain Fab fragments are paired with a light chain that is not the cognate light chain, or wherein the antibody comprises additional light chains. A “cognate pair” of an antibody heavy chain or heavy chain Fab fragment and a light chain denotes those chains that have been intended/selected/designed to pair to form the binding site with the correct binding properties with respect to a target.

### Recombinant Methods and Compositions

Antibodies may be produced using recombinant methods and compositions, e.g., as described in U.S. Pat. No. 4,816,567. For these methods, one or more isolated nucleic acid(s) encoding an antibody are provided.

In one aspect of the invention, a method of producing an antibody is provided, wherein the method comprises culturing a cell clone comprising nucleic acid(s) encoding the antibody, under conditions suitable for expression of the antibody, and optionally recovering the antibody from the host cell (or host cell culture medium), wherein the cell clone has been selected with a method according to the current invention.

For recombinant production of an antibody, nucleic acids encoding the antibody are generated/designed/synthesized and inserted into one or more vectors for further cloning and/or expression in a cell. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Generally, for the recombinant large-scale production of a polypeptide of interest, such as e.g. a therapeutic antibody, a cell clone stably expressing and secreting said polypeptide is required. This cell clone is termed “recombinant cell clone” or “recombinant production cell clone” and the overall process used for generating such a cell is termed “cell line development”. In the first step of the cell line development process, a suitable host cell, such as e.g., in certain embodiments, a CHO cell, is transfected with one or more nucleic acid sequences suitable for expression of said polypeptide of interest. In a second step, cell clones stably expressing the polypeptide of interest are selected based on the co-expression of a selection marker, which had been co-transfected with the nucleic acids encoding the polypeptide of interest.

A nucleic acid encoding a polypeptide, i.e. the coding sequence, is denoted as a structural gene. Such a structural gene is pure coding information. Thus, additional regulatory elements are required for expression thereof. Therefore, normally a structural gene is integrated in a so-called expression cassette. The minimal regulatory elements needed for an expression cassette to be functional in a mammalian cell are a promoter functional in said mammalian cell, which is located upstream, i.e. 5′, to the structural gene, and a polyadenylation signal sequence functional in said mammalian cell, which is located downstream, i.e. 3′, to the structural gene. The promoter, the structural gene and the polyadenylation signal sequence are arranged in an operably linked form.

In case the polypeptide of interest is a heteromultimeric polypeptide that is composed of different polypeptides, such as e.g. an antibody or a complex antibody format, not only a single expression cassette is required but a multitude of expression cassettes differing in the respectively contained structural gene, i.e. at least one expression cassette for each of the different polypeptides (chains) of the heteromultimeric polypeptide (heteromultimeric antibody). For example, a full-length antibody is a heteromultimeric polypeptide comprising two copies of a light chain as well as two copies of a heavy chain. Thus, a full-length antibody is composed of two different polypeptides. Therefore, two expression cassettes are required for the expression of a full-length antibody, one for the light chain and one for the heavy chain. If, for example, the full-length antibody is a bispecific antibody, i.e. the antibody comprises two different binding sites specifically binding to two different antigens/epitopes on the same antigen; the two light chains as well as the two heavy chains are also different from each other. Thus, such a bispecific, full-length antibody is composed of four different polypeptides and therefore, four expression cassettes are required.

The expression cassette(s) for the polypeptide of interest is(are) in turn integrated into one or more so called “expression vector(s)”. An “expression vector” is a nucleic acid providing all required elements for the amplification of said vector in bacterial cells as well as the expression of the comprised structural gene(s) in a mammalian cell. Typically, an expression vector comprises a prokaryotic plasmid propagation unit, e.g. for E. coli, comprising an origin of replication, and a prokaryotic selection marker, as well as a eukaryotic selection marker, and the expression cassettes required for the expression of the structural gene(s) of interest. An “expression vector” is a transport vehicle for the introduction of expression cassettes into a mammalian host cell to generate polypeptide-expressing cell clones.

As outlined in the previous paragraphs, the more complex the polypeptide to be expressed is, the higher also the number of required different expression cassettes will be. Inherently with the number of expression cassettes also the size of the nucleic acid to be integrated into the genome of the host cell increases. Concomitantly also the size of the expression vector increases. However, there is a practical upper limit to the size of a vector in the range of about 15 kbps above which handling and processing efficiency profoundly drops. This issue can be addressed by using two or more expression vectors. Thereby the expression cassettes can be split between different expression vectors each comprising only some of the expression cassettes resulting in a size reduction.

Cell line development (CLD) for the generation of recombinant cell expressing a heterologous polypeptide, such as e.g. a multispecific antibody, employs either random integration (RI) or targeted integration (TI) of the nucleic acid(s) comprising the respective expression cassettes required for the expression and production of the heterologous polypeptide of interest.

Using RI, in general, several vectors or fragments thereof integrate into the cell's genome at the same or different loci.

Using TI, in general, a single copy of the transgene comprising the different expression cassettes is integrated at a predetermined “hot-spot” in the host cell's genome.

Suitable host cells for the generation of cell clones for expression of an (glycosylated) antibody are generally derived from multicellular organisms such as e.g. vertebrates.

### Host Cells

Any mammalian host cell line that is adapted to grow in suspension can be used to generate recombinant cell clones that can be processed in the method according to the current invention. In addition, independent from the integration method, i.e. for RI as well as TI, any mammalian host cell can be used.

Examples of useful mammalian host cell lines are human amniocyte cells (e.g. CAP-T cells as described in Woelfel, J. et al., BMC Proc. 5 (2011) P133); monkey kidney CV1 line transformed by SV40 (COS-7); human embryonic kidney line (HEK293 or HEK293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

In certain embodiments, the mammalian host cell is, e.g., a Chinese Hamster Ovary (CHO) cell (e.g. CHO K1, CHO DG44, etc.), a Human Embryonic Kidney (HEK) cell, a lymphoid cell (e.g., Y0, NS0, Sp2/0 cell), or a human amniocyte cells (e.g. CAP-T, etc.). In one preferred embodiment, the mammalian (host) cell is a CHO cell. Thus, likewise the cell clone is a CHO cell.

With respect to TI, any known or future mammalian host cell suitable for TI comprising a landing site as described herein integrated at a single site within a locus of the genome can be used in the current invention. Such a cell is denoted as mammalian TI host cell. In certain embodiments, the mammalian TI host cell is a hamster cell, a human cell, a rat cell, or a mouse cell comprising a landing site as described herein. In one preferred embodiment, the mammalian TI host cell is a CHO cell. In certain embodiments, the mammalian TI host cell is a Chinese hamster ovary (CHO) cell, a CHO K1 cell, a CHO K1SV cell, a CHO DG44 cell, a CHO DUKXB-11 cell, a CHO K1S cell, or a CHO KIM cell comprising a landing site as described herein integrated at a single site within a locus of the genome.

In certain embodiments, a mammalian TI host cell comprises an integrated landing site, wherein the landing site comprises one or more recombination recognition sequence (RRS). The RRS can be recognized by a recombinase, for example, a Cre recombinase, an FLP recombinase, a Bxb1 integrase, or a φC31 integrase. The RRS can be selected independently of each other from the group consisting of a LoxP sequence, a LoxP L3 sequence, a LoxP 2L sequence, a LoxFas sequence, a Lox511 sequence, a Lox2272 sequence, a Lox2372 sequence, a Lox5171 sequence, a Loxm2 sequence, a Lox71 sequence, a Lox66 sequence, a FRT sequence, a Bxb1 attP sequence, a Bxb1 attB sequence, a φC31 attP sequence, and a φC31 attB sequence.

If multiple RRSs have to be present, the selection of each of the sequences is dependent on the other insofar as non-identical RRSs are chosen.

Although the invention is exemplified with a CHO cell hereafter, this is presented solely to exemplify the invention but shall not be construed in any way as limitation. The true scope of the invention is set forth in the claims.

### Targeted Integration

One method for the generation of a recombinant mammalian cell clone to be processed in the method according to the current invention is a recombinant cell clone generated by using targeted integration (TI) for the introduction of the coding nucleic acid.

In targeted integration, site-specific recombination is employed for the introduction of an exogenous nucleic acid into a specific locus in the genome of a mammalian TI host cell to generate recombinant cell clones. This is an enzymatic process wherein a sequence at the site of integration in the genome is exchanged for the exogenous nucleic acid. One system used to effect such nucleic acid exchanges is the Cre-lox system. The enzyme catalyzing the exchange is the Cre recombinase. The sequence to be exchanged is defined by the position of two lox(P)-sites in the genome as well as in the exogenous nucleic acid. These lox(P)-sites are recognized by the Cre recombinase. Nothing more is required, i.e. no ATP etc. Originally, the Cre-lox system has been found in bacteriophage P1.

The Cre-lox system operates in different cell types, like mammals, plants, bacteria and yeast.

In certain embodiments, the exogenous nucleic acid encoding the heterologous polypeptide has been integrated into the mammalian TI host cell by single or double recombinase mediated cassette exchange (RMCE). Thereby a recombinant mammalian cell clone, such as a recombinant CHO cell clone, is obtained, in which a defined and specific expression cassette sequence has been integrated into the genome at a single locus.

The Cre-LoxP site-specific recombination system has been widely used in many biological experimental systems. Cre recombinase is a 38-kDa site-specific DNA recombinase that recognizes 34 bp LoxP sequences. Cre recombinase is derived from bacteriophage P1 and belongs to the tyrosine family site-specific recombinase. Cre recombinase can mediate both intra and intermolecular recombination between LoxP sequences. The LoxP sequence is composed of an 8 bp non-palindromic core region flanked by two 13 bp inverted repeats. Cre recombinase binds to the 13 bp repeat thereby mediating recombination within the 8 bp core region. Cre-LoxP-mediated recombination occurs at a high efficiency and does not require any other host factors. If two LoxP sequences are placed in the same orientation on the same nucleotide sequence, Cre recombinase-mediated recombination will excise DNA sequences located between the two LoxP sequences as a covalently closed circle. If two LoxP sequences are placed in an inverted position on the same nucleotide sequence, Cre recombinase-mediated recombination will invert the orientation of the DNA sequences located between the two sequences. If two LoxP sequences are on two different DNA molecules and if one DNA molecule is circular, Cre recombinase-mediated recombination will result in integration of the circular DNA sequence.

A “recombination recognition sequence” (RRS) is a nucleotide sequence recognized by a recombinase and is necessary and sufficient for recombinase-mediated recombination events. A RRS can be used to define the position where a recombination event will occur in a nucleotide sequence.

The term “matching RRSs” indicates that a recombination occurs between two RRSs. In certain embodiments, the two matching RRSs are the same.

In certain embodiments, a RRS can be recognized by a Cre recombinase. In certain embodiments, a RRS can be recognized by a FLP recombinase. In certain embodiments, a RRS can be recognized by a Bxb1 integrase. In certain embodiments, a RRS can be recognized by a φC31 integrase.

In certain embodiments, both RRSs are wild-type LoxP sequences. In certain embodiments, both RRSs are mutant LoxP sequences. In certain embodiments, both RRSs are wild-type FRT sequences. In certain embodiments, both RRSs are mutant FRT sequences. In certain embodiments, the two matching RRSs are different sequences but can be recognized by the same recombinase. In certain embodiments, the first matching RRS is a Bxb1 attP sequence and the second matching RRS is a Bxb1 attB sequence. In certain embodiments, the first matching RRS is a φC31 attB sequence and the second matching RRS is a φC31 attB sequence.

A “two-plasmid RMCE” strategy or “double RMCE” is employed in the method according to the current invention when using a two-vector combination. For example, but not by way of limitation, an integrated landing site could comprise three RRSs, e.g., an arrangement where the third RRS (“RRS3”) is present between the first RRS (“RRS1”) and the second RRS (“RRS2”), while a first vector comprises two RRSs matching the first and the third RRS on the integrated exogenous nucleotide sequence, and a second vector comprises two RRSs matching the third and the second RRS on the integrated exogenous nucleotide sequence.

The two-plasmid RMCE strategy involves using three RRS sites to carry out two independent RMCEs simultaneously. Therefore, a landing site in the mammalian TI host cell using the two-plasmid RMCE strategy includes a third RRS site (RRS3) that has no cross activity with either the first RRS site (RRS1) or the second RRS site (RRS2). The two plasmids to be targeted require the same flanking RRS sites for efficient targeting, one plasmid (front) flanked by RRS1 and RRS3 and the other (back) by RRS3 and RRS2. In addition, two selection markers are needed in the two-plasmid RMCE. One selection marker expression cassette was split into two parts. The front plasmid would contain the promoter followed by a start codon and the RRS3 sequence. The back plasmid would have the RRS3 sequence fused to the N-terminus of the selection marker coding region, minus the start-codon (ATG). Additional nucleotides may need to be inserted between the RRS3 site and the selection marker sequence to ensure in frame translation for the fusion protein, i.e. operable linkage. Only when both plasmids are correctly inserted, the full expression cassette of the selection marker will be assembled and, thus, rendering cells resistance to the respective selection agent.

Two-plasmid RMCE involves double recombination crossover events, catalyzed by a recombinase, between the two heterospecific RRSs within the target genomic locus and the donor DNA molecule. Two-plasmid RMCE is designed to introduce a copy of the DNA sequences from the front- and back-vector in combination into the pre-determined locus of a mammalian TI host cell's genome. RMCE can be implemented such that prokaryotic vector sequences are not introduced into the mammalian TI host cell's genome, thus, reducing and/or preventing unwanted triggering of host immune or defense mechanisms. The RMCE procedure can be repeated with multiple DNA sequences.

In certain embodiments, targeted integration is achieved by two RMCEs, wherein two different DNA sequences, each comprising at least one expression cassette encoding a part of a heteromultimeric polypeptide and/or at least one selection marker or part thereof flanked by two heterospecific RRSs, are both integrated into a pre-determined site of the genome of a RRSs matching mammalian TI host cell. In certain embodiments, targeted integration is achieved by multiple RMCEs, wherein DNA sequences from multiple vectors, each comprising at least one expression cassette encoding a part of a heteromultimeric polypeptide and/or at least one selection marker or part thereof flanked by two heterospecific RRSs, are all integrated into a predetermined site of the genome of a mammalian TI host cell. In certain embodiments the selection marker can be partially encoded on the first the vector and partially encoded on the second vector such that only the correct integration of both by double RMCE allows for the expression of the selection marker.

In certain embodiments, targeted integration via recombinase-mediated recombination leads to selection marker and/or the different expression cassettes for the multimeric polypeptide integrated into one or more pre-determined integration sites of a host cell genome free of sequences from a prokaryotic vector.

An exemplary mammalian TI host cell that is suitable for use in a method according to the current invention is a CHO cell harboring a landing site integrated at a single site within a locus of its genome wherein the landing site comprises three heterospecific loxP sites for Cre recombinase mediated DNA recombination.

In this example, the heterospecific loxP sites are L3, LoxFas and 2L (see e.g. Lanza et al., Biotechnol. J. 7 (2012) 898-908; Wong et al., Nucleic Acids Res. 33 (2005) e147), whereby L3 and 2L flank the landing site at the 5′-end and 3′-end, respectively, and LoxFas is located between the L3 and 2L sites.

Such a configuration of the landing site as outlined in the previous paragraph allows for the simultaneous integration of two vectors, e.g. of a so called front vector harboring an L3 and a LoxFas site and a back vector harboring a LoxFas and an 2L site. The functional elements of a selection marker gene different from that present in the landing site can be distributed between both vectors: promoter and start codon can be located on the front vector whereas coding region and poly A signal are located on the back vector. Only correct recombinase-mediated integration of said nucleic acids from both vectors induces resistance against the respective selection agent.

Generally, a mammalian TI host cell is a mammalian cell comprising a landing site integrated within a locus of the genome of the mammalian cell, wherein the landing site comprises a first and a second recombination recognition sequence flanking at least one first selection marker, and a third recombination recognition sequence located between the first and the second recombination recognition sequence, and all the recombination recognition sequences are different.

An exogenous nucleotide sequence is a nucleotide sequence that does not originate from a specific cell but can be introduced into said cell by DNA delivery methods, such as, e.g., by transfection, electroporation, or transformation methods. In certain embodiments, a mammalian TI host cell comprises at least one landing site integrated at one or more integration sites in the mammalian cell's genome. In certain embodiments, the landing site is integrated at one or more integration sites within a specific a locus of the genome of the mammalian cell.

In certain embodiments, the integrated landing site comprises at least one selection marker. In certain embodiments, the integrated landing site comprises a first, a second and a third RRS, and at least one selection marker. In certain embodiments, a selection marker is located between the first and the second RRS. In certain embodiments, two RRSs flank at least one selection marker, i.e., a first RRS is located 5′ (upstream) and a second RRS is located 3′ (downstream) of the selection marker. In certain embodiments, a first RRS is adjacent to the 5′-end of the selection marker and a second RRS is adjacent to the 3′-end of the selection marker. In certain embodiments, the landing site comprises a first, second, and third RRS, and at least one selection marker located between the first and the third RRS.

In certain embodiments, a selection marker is located between a first and a second RRS and the two flanking RRSs are different. In certain preferred embodiments, the first flanking RRS is a LoxP L3 sequence and the second flanking RRS is a LoxP 2L sequence. In certain embodiments, a LoxP L3 sequenced is located 5′ of the selection marker and a LoxP 2L sequence is located 3′ of the selection marker. In certain embodiments, the first flanking RRS is a wild-type FRT sequence and the second flanking RRS is a mutant FRT sequence. In certain embodiments, the first flanking RRS is a Bxb1 attP sequence and the second flanking RRS is a Bxb1 attB sequence.

In certain embodiments, the first flanking RRS is a cφC31 attP sequence and the second flanking RRS is a φC31 attB sequence. In certain embodiments, the two RRSs are positioned in the same orientation. In certain embodiments, the two RRSs are both in the forward or reverse orientation. In certain embodiments, the two RRSs are positioned in opposite orientation.

### Description of Specific Embodiments of the Method of the Invention

The method according to the current invention is exemplified in the following for bispecific antibody formats using a set of four ELISA-based assays (one for determining the antibody titer in the supernatant of the cultivation of one cell clone expressing said bispecific antibody, two for determining the isolated binding to antigen 1 and antigen 2, respectively, of the bispecific antibody, and one for determining the simultaneous binding to antigen 1 and antigen 2). The readouts thereof are processed based on the method according to the current invention. Thereby on the one hand, cell clones can be differentiated based on the ratio of the antibody main product to antibody side product and on the other hand, cell clones can be correctly classified with respect to their product(antibody)-related side product profile. Both provide the basis for an improved clone selection process according to the method of the current invention. This example is presented solely as an exemplification of the method according to the current invention and shall not be construed as a limitation thereof. The true scope of the invention is set forth in the appended claims.

In this example, the assessment of individual cell clones producing a bispecific antibody is based on the results of four assays, with which the antibody titer in the supernatant, the individual binding to antigens 1 and 2, respectively, and the simultaneous binding to both antigens is determined and quantified.

Thus, for cell clone selection during cell line development (CLD) the different cell clones obtained by the transfection of a host cell with one or more nucleic acids encoding the bispecific antibody are analyzed by the three binding ELISAs as outlined above and are rated according to desired antibody main product vs. undesired antibody side product amounts (=antibody main product quality) in view of the expression yield. The combined readout of these assays allows the discrimination of clones expressing a high percentage of the desired antibody main product (=lead) from clones expressing only a low percentage of desired main product.

Many bispecific antibody formats are based on the knob-into-hole heterodimerization technology. Such formats can form characteristic product-related side products, e.g., due to chain mispairings, such as, e.g., hole chain-hole chain dimers, knob chain-knob chains dimers, or % antibodies missing one light chain. Different cell clones often express different mixtures of the antibody main product (=lead, i.e. correctly assembled bispecific antibody) and product-related antibody side products (e.g., hole chain-hole chain dimers (=side-product I) and knob chain-knob chain dimers (=side product II)). The differences in the expression pattern of these molecules of the recombinant cell clones require an early and high throughput-suited screening method to deselect cell clones with unsuitable product profile, such as those with, e.g., low antibody main product yield. The antibody formats used in this example and some of their side-products are depicted in FIG. 15.

The assays employed in this example of the method according to the current invention utilize different assay principles. This results in the effect that the different expression products give different ELISA signals. The purified main product (i.e. the correctly assembled knob-into-hole heterodimer) and the most common side products (i.e. the hole-hole homodimer and the knob-knob homodimer) are used as standards. They result in specific signals in the assays based on avidity effects a) by the format itself and b) due to the employed detection antibody.

For determining antigen binding, the following enzyme-linked immunosorbent assays (ELISAs) were used in this example:
biotinylated antigen 1 or 2, respectively, was immobilized on a streptavidin coated microtiter plate; after washing the plate, a mixture of cultivation supernatant and detection antibody (anti-human Fab antibody conjugated to POD (horseradish peroxidase)) was applied; 3,3′,5,5′-Tetramethylbenzidine (TMB) was used for the readout;
simultaneous binding to antigen 1 and antigen 2 was determined in a bridging ELISA; antigen 1 or antigen 2 was directly coated on a microtiter plate; after washing the plate, a mixture of cultivation supernatant, the respective other biotinylated antigen 2 or 1, respectively, and a streptavidin-POD conjugate was applied; TMB was used for the readout.

These ELISA readouts were combined according to the method according to the current invention to differentiate antibody main product, i.e. monomeric and correctly assembled bispecific antibody, and antibody side products. Thereby a classification of the respective clones regarding to their properties as basis for clone selection has been achieved. For example, a clone is classified as “Lead” if it is predicted to predominantly express the main product based on the application of the method according to the current invention. Such clones are suitable as clones for expressing/producing the bispecific antibody with low antibody-related side product contents. These clones need to be identified.

As a reference, the categorization of the ELISA results for three different bispecific antibody formats—a domain exchanged 1+1 bispecific antibody (CrossMab), an N-terminal Fab-domain inserted 2+1 bispecific antibody (TCB) and C-terminal Fab domain fused 2+1 bispecific antibody (BS)—are visualized in FIGS. 1 to 3. The ELISA results are only shown for two out of three ELISAs and only for one out of four analyzed concentrations for clarity reasons. In the Figures, binding to antigen 2 (AG 2) is plotted against binding to antigen 1 (AG 1). The different shadings indicate the cell clone quality categorization. This shows that there is a variance depending on the antibody format and the target and/or target combinations (=project) and therefore demonstrates the complexity of the analysis.

The purified antibody main product (denoted as “main product standard”; e.g. the correctly assembled knob-into-hole heterodimeric heavy chain pairing with all light chains correctly associated) and the most common undesired side-products (denoted as “side-product I standard” and “side-product II standard”, respectively; e.g. the hole-hole homodimer and the knob-knob homodimer, respectively) were used as standards in all assays in a dilution series (14 different concentrations). The respective single cell clone cultivation supernatants after 5 days of cultivation were analyzed in 4 different concentrations. The method according to the current invention can be performed independent of the absolute signal differences, as long as these differences are statistically significant. This is independent of the signal being within the linear signal range of the assays or not. In certain embodiments, all standards, cultivation supernatants, coated antigens, detection antibody and detection reagents are used in concentrations, which result in maximal signal difference between the signal obtained with the antibody main product standard and the signal obtained with the antibody side product standards. A person skilled in the art can easily perform such an optimization of the signal difference based on his knowledge in the art without undue burden.

The invention is based, at least in part, on the finding that the selection of a recombinant cell clone expressing a bispecific antibody can be efficiently done based on the concentration of the bispecific antibody in single cell clone cultivation supernatant and the results of immunoassays determining the binding of the bispecific antibody to the isolated antigens as well as the simultaneous binding to both antigens. It has further been found that a purposive data processing is advantageous.

The invention is based, at least in part, on the finding that the concentration of a bispecific antibody in a single cell clone cultivation supernatant (after at least 5 days of cultivation) in combination with
the median blank-corrected data (binding signal) of four measured concentrations of the cultivation supernatant in binding immunoassays for the isolated binding to each antigen and the simultaneous binding to both antigens,
the median blank-corrected data of at least five measured concentrations of the isolated bispecific antibody and the two main side products in each of the binding immunoassays,

can be used for the selection of recombinant cell clones producing a bispecific antibody with the lowest antibody-related side product content.

The terms “blank-corrected data” or “blank-corrected binding signal”, which are used interchangeably herein, denote that from the binding signal determined in an immunoassay for a test or standard sample, i.e. from the raw binding signal determined for the test or standard sample, the background signal determined in the same immunoassay for a blank sample, i.e. a sample without standard, cultivation supernatant, (therapeutic) antibody and any side product but otherwise identical to the test sample, is/has been subtracted.

It has been found that by using either all nine or four out of the nine possible standard-normalized binding ratios, and optionally the ratio of the absorbance corrected ELISA signals the method according to the current invention can be further improved.

The invention is based, at least in part, on the finding that certain ratios of the binding signals are specific and allow, when used in combination and normalized with/to the ratios calculated for the standards (100% purified antibody main product, antibody side-product I and antibody side-product II), the differentiation of individual cultivation supernatants (=samples) and thereby of clones with good (mainly producing/expressing antibody main product) from clones with poor (mainly producing/expressing antibody side-products) product quality.

The ratios for the standards are calculated as medians of the signals obtained for the concentrations (in one preferred embodiment for 4 concentrations of the cultivation supernatant and for 14 concentrations of the standards) and are then divided by each other (“normalization”). This leads to a total of nine possible ratios.

The inventions is based, at least in part on the finding that only the following four medians are required for the selection of cell clones expression correctly assembled bispecific antibody:
median of the ratios of the blank-corrected binding signals obtained for the same (four) concentrations of the cell clone cultivation supernatant for binding to antigen 2 to the blank-corrected binding signal obtained for the same (four) concentrations of the cell clone cultivation supernatant for binding to antigen 1, which is normalized to (i.e. divided by) the median of the ratios of the blank-corrected binding signals obtained for at least 5 concentrations of the antibody main product (main product standard) for binding to antigen 2 to the blank-corrected binding signal obtained for the same at least 5 concentrations of main product standard for binding to antigen 1, determined in the same assays,
median of the ratios of the blank-corrected binding signals obtained for the same (four) concentrations of the cell clone cultivation supernatant for simultaneous binding to antigen 1 and 2 to the blank-corrected binding signal obtained for the same (four) concentrations of the cell clone cultivation supernatant for binding to antigen 1, which is normalized to the median of the ratios of the blank-corrected binding signals obtained for at least 5 concentrations of the antibody main product (main product standard) for simultaneous binding to antigen 1 and 2 to the blank-corrected binding signal obtained for the same at least 5 concentrations of the main product standard for binding to antigen 1, determined in the same assays,
median of the ratios of the blank-corrected binding signals obtained for the same (four) concentrations of the cell clone cultivation supernatant for binding to antigen 2 to the blank-corrected binding signal obtained for the same (four) concentrations of the cell clone cultivation supernatant for binding to antigen 1, which is normalized to the median of the ratios of the blank-corrected binding signals obtained for at least 5 concentrations of the antibody-related side product I (side product I standard) for binding to antigen 2 to the blank-corrected binding signal obtained for the same at least 5 concentrations of the side product I standard for binding to antigen 1, determined in the same assay, and
median of the ratios of the blank-corrected binding signals obtained for the same (four) concentrations of the cell clone cultivation supernatant for simultaneous binding to antigens 1 and 2 to the blank-corrected binding signal obtained for the same (four) concentrations of the cell clone cultivation supernatant for binding to antigen 1, which is normalized to the median of the ratios of the blank-corrected binding signals obtained for at least 5 concentrations of the antibody-related side product II (side product II standard) for simultaneous binding to antigen 1 and 2 to the blank-corrected binding signal obtained for the same at least 5 concentrations of the side product II standard for binding to antigen 1, determined in the same assay.

In certain embodiments, at least 10 concentrations of the respective standard is used. In one preferred embodiment, at least 14 concentrations of the respective standard is used.

When using at least these parameters the complexity of the cell clone characterization can be reduced. The resulting method according to the current invention, thus, encompasses this improved data processing and evaluation.

It has been found that by using the above selection of parameters in combination with an in silico analysis an antibody format-independent as well as target-independent method has been obtained.

To show the advantageous properties of the method according to the current invention, eight different exemplary data processing variants have been compared. These are summarized in the following Table.

| | number | |
|---|---|---|
| processing | standard | |
| variant no | concentrations | analyzed dataset includes |
| 1 | not applicable | titer, sample data |
| 2 | 4 | titer, sample data, standards in 4 concentrations |
| 3 | 4 | titer, sample data, 1-standard-normalized samples |
| 4 | 4 | titer, sample data, 3-standard-normalized samples |
| 5 | 4 | titer, sample data, ratios, 9 normalized ratios |
| 6 | 14 | titer, sample data, ratios, 9 normalized ratios |
| 7 | 14 | titer, sample data, 9 normalized ratios |
| 8 | 14 | titer, sample data, 4 normalized ratios |

| | |
|---|---|
| titer | total antibody titer, determined by ELISA using 8 dilutions of |
| | the sample |
| sample | supernatant of respective cell clone |
| sample data | absorbance corrected ELISA signals for 4 sample |
| | concentrations in 3 different ELISA assays (binding to AG1, |
| | binding to AG2, bridging of AG1 and AG2) |
| standards | purified (100%) antibody main product, side-product I and |
| | side-product II, respectively |
| standards in 4 | standards are evaluated using mean of 4 analyzed sample |
| concentrations | concentrations |
| standards in 14 | standards are evaluated using average of 14 analyzed sample |
| concentrations | concentrations |
| normalized | calculated ratio to standard |
| 1-standard- | sample data normalized to main product standard |
| normalized | |
| samples | |
| 3-standard | sample data normalized to main product, side-product I and |
| normalized | side-product II standards |
| samples | |
| ratios | ratio of sample data AG2 to AG1, AG12 to AG1, and AG12 |
| | to AG2 |
| 3-standard- | ratios normalized to main product, side-product I and side- |
| normalized | product II standards |
| ratios | |
| 9 normalized | AG2:AG1, AG12:AG1 and AG12:AG2 ratios each |
| ratios | normalized to main product, side-product I and side-product II |
| | standards |
| 4 normalized | AG2:AG1 ratio normalized to main product standard, |
| ratios | AG12:AG1 ratio normalized to main product standard, |
| | AG2:AG1 ratio normalized to side-product I standard, |
| | AG12:AG1 normalized to side-product II standard |

The respective performance of these eight different processing variants in an in silico processing with an Extreme Gradient Boosting (XGB) model regarding quality category predictions for five different target combinations in different formats are visualized in FIG. 4 (TCB-1, 2+1 Head-to-Tail, TCB-2, TCB-3) and CrossMab). The XGB model predictive quality was assessed by cross-entropy loss on an entire project not utilized for model training. The different processing methods as shown in the previous Table were used as experimental conditions.

In FIG. 5 the false negative as well as the false positive rates for the different processing variants are shown. The effect of the different combinations of input parameters and engineered features can be clearly seen, whereby processing variants 6, 7 and 8 perform best.

Thus, with the method according to the current invention a target- and format-independent recombinant cell clone categorization can be achieved.

The quality of the method according to the current invention is shown in FIGS. 6 to 10. In these Figures a dataset evaluated using a manual-only method is compared with the same dataset evaluated with the method according to the current invention. The project, which was used to demonstrate the improved performance of the method according to the current invention compared to the manual-only evaluation method, has not been used in the training of the algorithm. Therefore, a bias can be excluded.

In FIG. 6 the binding to antigen 2 (AG2) is plotted against the binding to antigen 1 (AG1). The manual-only analysis is shown in the top and the results obtained with the method according to the current invention is shown in the bottom. FIG. 7 to 10 depict in the same arrangement the medians of the ratios AG2:AG1 normalized to main product standard, AG12:AG1 normalized to main product standard, AG2:AG1 normalized to side-product I standard, and AG12:AG2 normalized to side-product II standard, each plotted against the clone number. Due to the normalization against the standards, clones falling into the respective categorization should have values close to 1. The improved precision of the results obtained with the method according to the current invention is shown impressively in these graphs. The shading indicates the different quality categories, while the size in the bottom datasets is proportional to the prediction precision and, thus, shows very easily and fast, which clone has the highest likelihood to produce an excellent product quality.

The method according to the current invention can be used for clone quality assessment to select clones with the best properties for upscaling as shown in the next example.

In FIGS. 11 to 14 an example for a clone selection with a method according to the current invention is shown. The clone quality categories determined with the method according to the current invention are shown in FIGS. 11 and 12 by the different shadings. In FIGS. 13 and 14 the selection process is demonstrated. 160 out of the 610 clones were selected based on the result of the method according to the current invention to have the desired product quality, i.e. antibody main product yield.

The cross-entropy loss function (logarithmic loss, log loss or logistic loss) is a function for ranking data in a model. Each predicted class probability is compared to the actual class desired output 0 or 1 and a score/loss is calculated that penalizes the probability based on how far it is from the actual expected value. The penalty is logarithmic in nature yielding a large score for large differences close to 1 and small score for small differences tending to 0. Cross-entropy loss is used when adjusting model weights during training. The aim is to minimize the loss, i.e., the smaller the loss the better the model. A perfect model has a cross-entropy loss of 0. Cross-entropy is defined as

$${L}_{C E} = - {\sum }_{i = 1}^{n} {t}_{i} * log ( {p}_{i} )$$

for n classes, wherein tᵢ is the truth label and pi is the probability for the i^{th} class.

The median is the term of a series of terms that is in the middle of said series. For a series with N terms, wherein N is an odd number, the median is the

$$\frac{{( N + 1 )}^{t h}}{2}$$

term, i.e. the term in the middle of the series. For a series with N terms, wherein N is an even number, the media is the

$$\frac{( {( \frac{N}{2} )}^{t h} + {( \frac{N}{2} + 1 )}^{t h} ) term}{2} ,$$

i.e. the average of the two terms in the middle of the series. The series comprises the individual terms in ascending numerical (value) order.

By using the method according to the current invention, the number of clones producing a product with poor product quality selected during early steps of clone selection is reduced, i.e. the number of false positives is reduced, and at the same time in addition the loss, i.e. the deselection, of clones producing a product with good product quality is also reduced, i.e. the number of false negatives is reduced.

When using state of the art screening methods not including product quality attributes in the selection of clones, clones producing a product with poor product quality will only be recognized late in the screening process, e.g. once sufficient product is available so that quality can be analyzed by analytical chromatography.

Thus, by reducing the number of false positive clones more good producing clones, i.e. true positives, are carried further in the selection process. Thereby the potential loss of clones producing a product of high quality is reduced.

This is nicely shown in FIGS. 16 to 20.

In FIG. 16 each bar represents one clone. The clones are ranked by titer, i.e. product concentration, size of the bar). In addition, the clones have been characterized with the method according to the invention. Clones that would have been selected with the method according to the invention are shown in dark.

Using a state-of the art titer-based selection a combination of light and dark bars would have been selected, whereas with the method according to the invention only the clones represented by the dark bars would have been selected.

Thus, using state of the art, titer-based selection clones not producing a high quality product will be included (light bars), i.e. false positives, and thereby the number of selected true positive clones is reduced.

All clones, i.e. the product produced by them, have been analyzed by size-exclusion chromatography (SEC) to determine monomer content as well as by capillary electrophoresis (CE-SDS) and hydrophobic interaction chromatography (HIC) for determining main product peak.

In FIG. 17 the relation between SEC-determined monomer content (x-axis) and amount of main product determined by CE-SDS (y-axis) is shown for clones of FIG. 16. That is what is analyzed in early clone selection. Clones that would have been deselected with the method according to the invention, i.e. the light bars of FIG. 16, are shown in bold. It can be seen that approximately half of the bold deselected clones can be found in the upper right quadrant, i.e. having based on CE-SDS and SEC an allegedly good product quality (circle in FIG. 17). However, these are according to the method according to the invention false positives.

This has been confirmed re-analyzing the clones from the circle of FIG. 17 using HIC. The result is shown in FIG. 18. The encircled, bold clones of the upper right quadrant of FIG. 17 are now found in the lower right quadrant in FIG. 18 (circle), i.e. have a low main product peak and, thus, are false positives.

To further confirm the advantageous properties of the method according to the current invention, the same analysis has been done for clones that are selected by the method according to the current invention. The corresponding results for SEC in combination with CE-SDS are shown in FIG. 19 and for SEC in combination with HIC in FIG. 20, respectively.

It can be seen that clones selected with the method according to the current invention are located in both Figures in the upper right quadrant and, thus, are true positive clones.

This shows that with the method according to the current invention the number of selected false positive clones can be reduced and the same time the number of deselected true positive clones can be increased.

### Description of Specific Computer-Implemented Embodiments of the Invention

One aspect of the current invention is a computer program product comprising instructions that, when executed on a suitable system comprising a computer, an immunoassay measurement device, causing at least the following steps to be performed
i) determining a signal proportional to the antibody concentration in a single cell clone cultivation supernatant for different concentrations of the single cell clone cultivation supernatant;
ii) determining a signal proportional to the isolated binding of the antibody contained in the single cell cultivation supernatant to its first target for different concentrations of the single cell clone cultivation supernatant;
iii) determining a signal proportional to the isolated binding of the antibody contained in the single cell cultivation supernatant to its second target for different concentrations of the single cell clone cultivation supernatant;
iv) determining a signal proportional to the simultaneous binding of the antibody contained in the single cell cultivation supernatant to its first and second target for different concentrations of the single cell clone cultivation supernatant;
v) determining a signal proportional to the isolated binding of the antibody reference standard, the antibody-related side product I standard and the antibody-related side product II standard to the first target of the antibody for different concentrations of the respective standards;
vi) determining a signal proportional to the isolated binding of the antibody reference standard, the antibody-related side product I standard and the antibody-related side product II standard to the second target of the antibody for different concentrations of the respective standards;
vii) determining a signal proportional to the simultaneous binding of the antibody reference standard, the antibody-related side product I standard and the antibody-related side product II standard to the first and second target of the antibody for different concentrations of the respective standards.

In certain embodiments, the instructions further include after step vii) the following steps
a) calculating the median of the ratio of the signals obtained for the same four concentrations of the cell clone cultivation supernatant for binding to antigen 2 to the signal obtained for the same four concentrations of the cell clone cultivation supernatant for binding to antigen 1, which is normalized to the median signal of the respective ratios obtained for at least 5 concentrations of the antibody main product (main product standard),
b) calculating the median of the ratio of the signals obtained for the same four concentrations of the cell clone cultivation supernatant for simultaneous binding to antigens 1 and 2 to the signal obtained for the same four concentrations of the cell clone cultivation supernatant for binding to antigen 1, which is normalized to the median signal of the respective ratios obtained for at least 5 concentrations of the antibody main product (main product standard),
c) calculating the median of the ratio of the signals obtained for the same four concentrations of the cell clone cultivation supernatant for binding to antigen 2 to the signal obtained for the same four concentrations of the cell clone cultivation supernatant for binding to antigen 1, which is normalized to the median signal of the respective ratios obtained for at least 5 concentrations of the antibody-related side product I (side product I standard), and
d) calculating the median of the ratio of the signals obtained for the same four concentrations of the cell clone cultivation supernatant for simultaneous binding to antigens 1 and 2 to the signal obtained for the same four concentrations of the cell clone cultivation supernatant for binding to antigen 1, which is normalized to the median signal of the respective ratios obtained for at least 5 concentrations of the antibody-related side product II (side product II standard).

In certain embodiments, the instructions further include fitting the calculated median values and the determined titer values of i) to a model for quality category prediction of recombinant cell clones expressing a bispecific antibody. In one preferred embodiment, the model is am XGB model.

In certain embodiments, the system further comprises a clone picking device and the final step viii) the step of selecting clones based on the smallest difference with respect to the concentration, the binding signals and the bispecific antibody divided ratios in combination with the biggest difference with respect to the side product divided ratios.

A further aspect of the current invention is a computer program including computer-executable instructions for performing the method according to the current invention in one or more of the embodiments enclosed herein when the program is executed on a computer or computer network. Specifically, the computer program may be stored on a computer-readable data carrier. Thus, specifically, one, more than one or even all of method steps i) to vii) and optionally one, more than one or even all of the method steps a) to d) and further optionally step viii) as indicated above may be performed by using a computer or a computer network, preferably by using a computer program.

A further aspect of the current invention is a computer program product having program code means, in order to perform the method according to the current invention in one or more of the embodiments enclosed herein when the program is executed on a computer or computer network. Specifically, the program code means may be stored on a computer-readable data carrier.

A further aspect of the current invention is a data carrier having a data structure stored thereon, which, after loading into a computer or computer network, such as into a working memory or main memory of the computer or computer network, may execute the method according to the invention in one or more of the embodiments thereof.

A further aspect of the current invention is a computer program product with program code means stored on a machine-readable carrier, in order to perform the method according to the invention in one or more of its embodiments, when the program is executed on a computer or computer network.

As used herein, a “computer program product” refers to the program as a tradable product. The product may generally exist in an arbitrary format, such as in a paper format, or on a computer-readable data carrier. Specifically, the computer program product may be distributed over a data network.

Finally, a further aspect according to the current invention is a modulated data signal that contains instructions readable by a computer system or computer network, for performing the method according to the invention in one or more of its embodiments.

Referring to the computer-implemented aspects of the invention, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Specifically, further disclosed herein are:
a computer or computer network comprising at least one processor, wherein the processor is adapted to perform the method according to the invention in one of its embodiments,
a computer loadable data structure that is adapted to the method according to the invention in one of its embodiments while the data structure is being executed on a computer,
a computer program, wherein the computer program is adapted to perform the method according to the invention in one of its embodiments while the program is being executed on a computer,
a computer program comprising program means for performing the method according to the invention in one of its embodiments while the computer program is being executed on a computer or on a computer network,
a computer program comprising program means according to any of the embodiment presented herein, wherein the program means are stored on a storage medium readable to a computer,
a storage medium, wherein a data structure is stored on the storage medium and wherein the data structure is adapted to perform the method according to the invention in one of its embodiments after having been loaded into a main and/or working storage of a computer or of a computer network, and
a computer program product having program code means, wherein the program code means can be stored or are stored on a storage medium, for performing the method according to the invention in one of its embodiments, if the program code means are executed on a computer or on a computer network.

The following examples and figures are provided to aid the understanding of the present invention, the true scope of which is set forth in the appended claims.

It is understood that modifications can be made in the procedures set forth without departing from the spirit of the invention.

### DESCRIPTION OF THE FIGURES

FIG. 1 Exemplary categorization of antigen binding ELISA readouts (AG2 vs. AG1) for a bispecific antibody in CrossMab format for a single concentration.

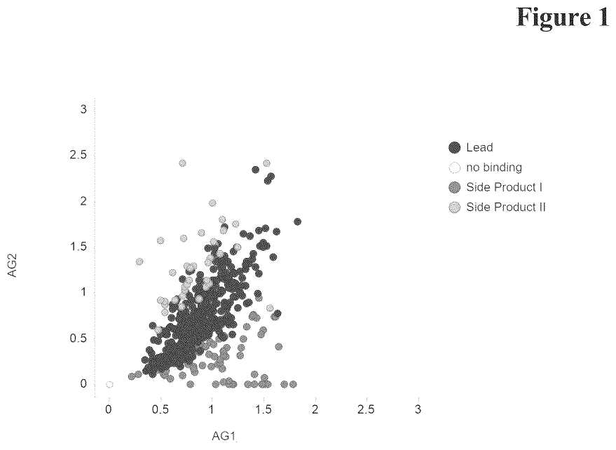

FIG. 2 Exemplary categorization of antigen binding ELISA readouts (AG 2 vs. AG 1) for a bispecific antibody in TCB format for a single concentration.

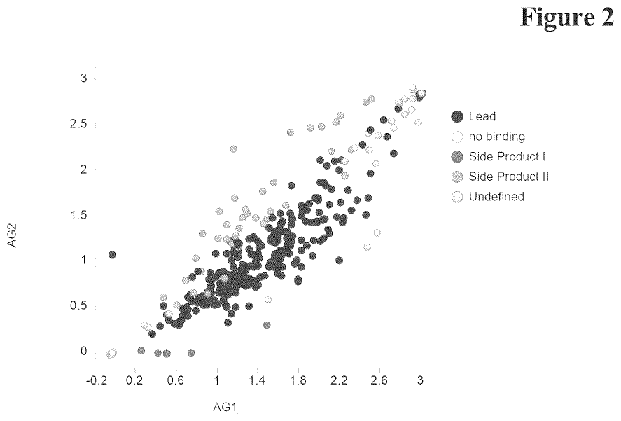

FIG. 3 Exemplary categorization of antigen binding ELISA readouts (AG2 vs. AG1) for a bispecific antibody in BS format for a single concentration.

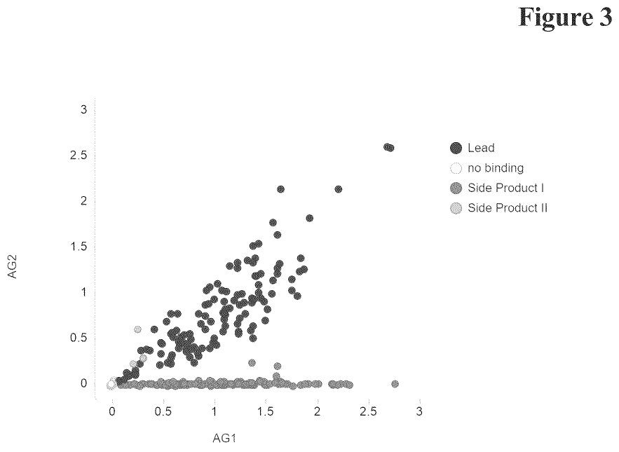

FIG. 4 Model predictive quality as assessed by cross-entropy loss (y-axis) on an entire project not utilized for model training. For each pre-processing variant 1 to 8 from left to right for TCB-1; 2+1 Head-to-Tail; TCB-2; TCB-3; CrossMab.

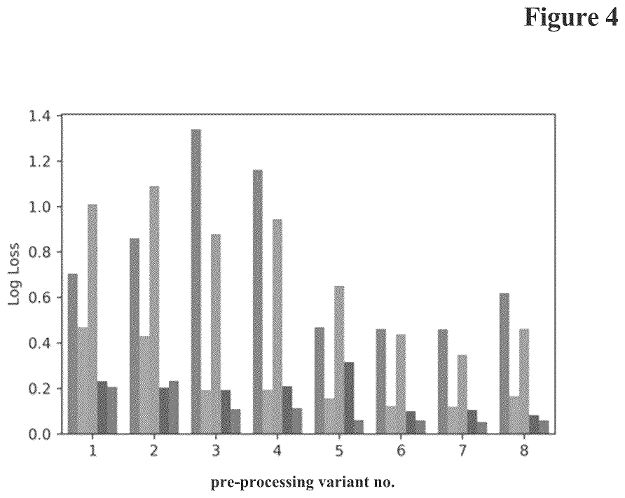

FIG. 5 False positive and false negative predictions [%] for being categorized as “correctly assembled bispecific antibody” across all five analyzed projects.

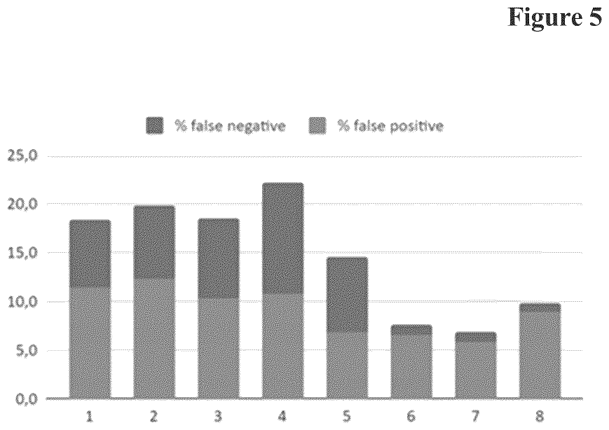

FIG. 6 Comparison of manual-only analysis (top) and the method according to the current invention (bottom). Binding to antigen 2 (AG2) is plotted against the binding to antigen 1 (AG1) in the top graph. The Figure shows the data for 610 monoclonal cell lines from a single project, which have not been used to train the algorithm. The size indicates the prediction precision.

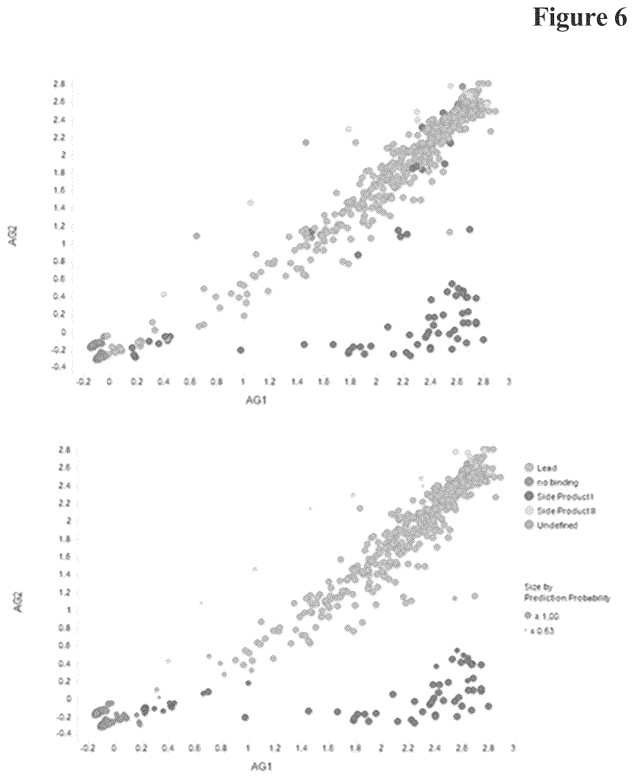

FIG. 7 Comparison of manual-only analysis (top) and the method according to the current invention (bottom). Median of ratios AG2:AG1 binding signal normalized to median of main product standard ratios AG2:AG1 binding signal plotted against the clone number.

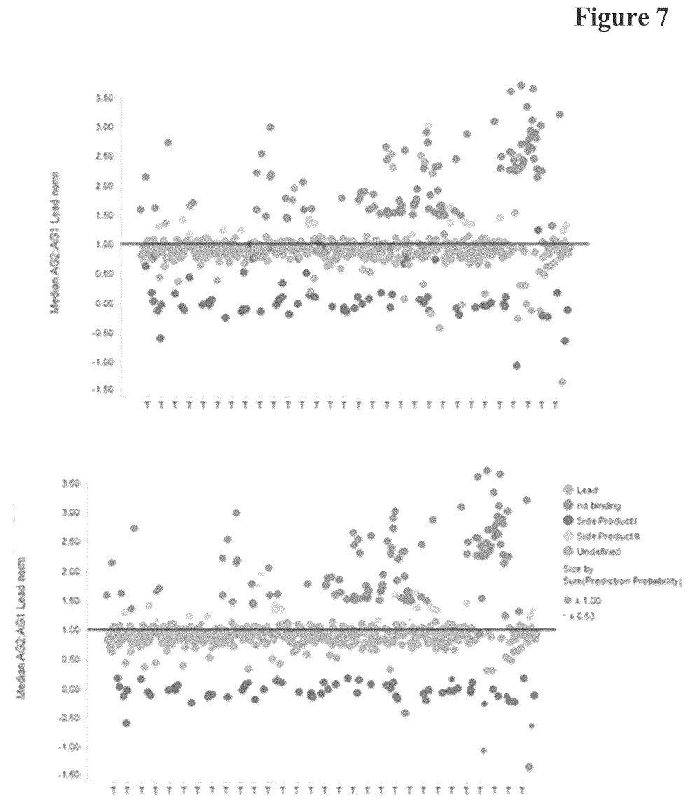

FIG. 8 Comparison of manual-only analysis (top) and the method according to the current invention (bottom). Median of ratios AG12:AG1 binding signal normalized to median of main product standard ratios AG12:AG1 binding signal plotted against the clone number.

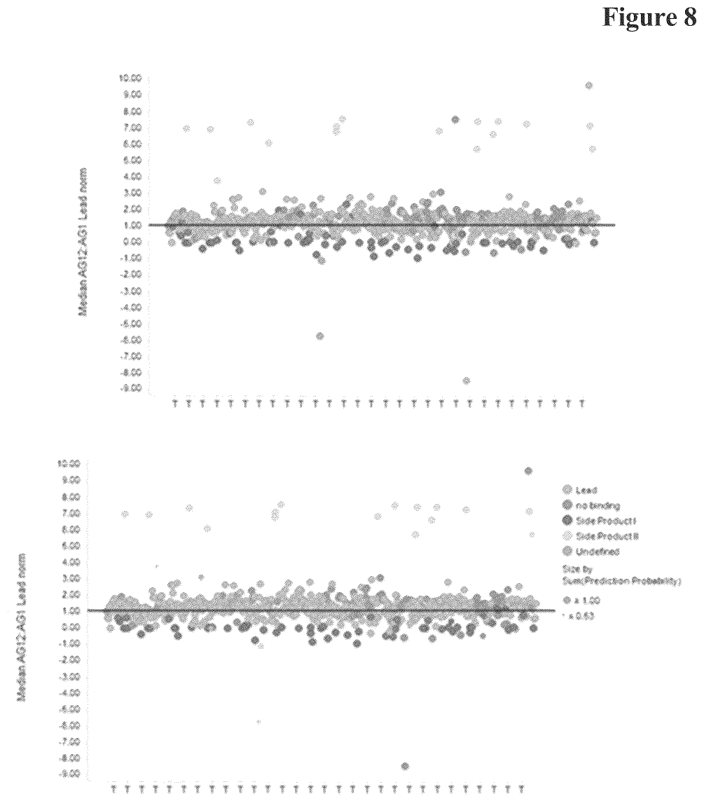

FIG. 9 Comparison of manual-only analysis (top) and the method according to the current invention (bottom). Median of ratios AG2:AG1 binding signal normalized to median of side product I standard ratios AG2:AG1 binding signal plotted against the clone number.

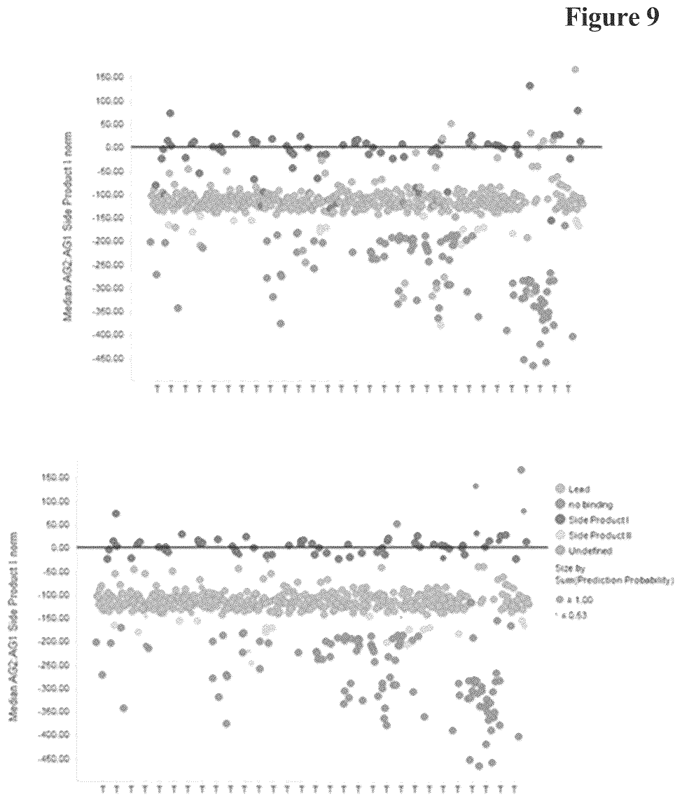

FIG. 10 Comparison of manual-only analysis (top) and the method according to the current invention (bottom). Median of ratios AG12:AG1 binding signal normalized to median of side product II standard ratios AG2:AG1 binding signal plotted against the clone number.

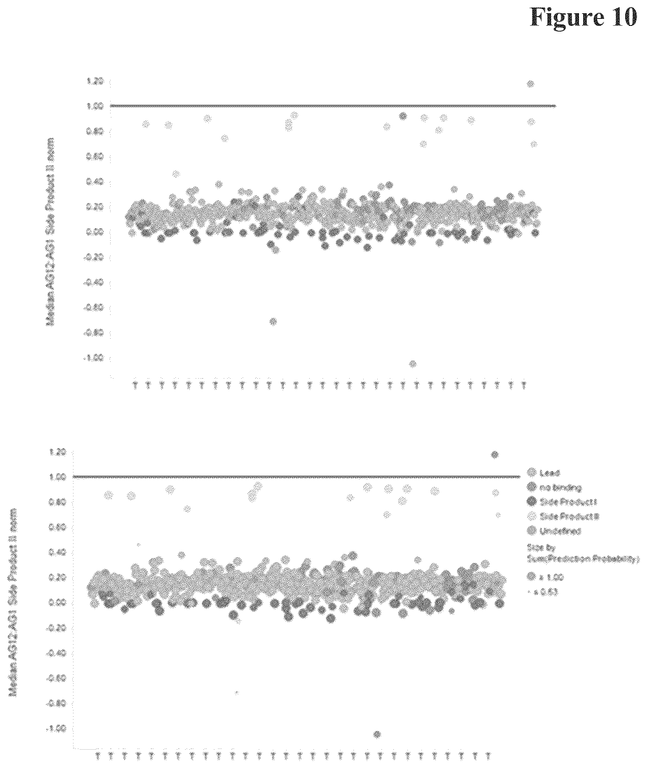

FIG. 11 Use of the method according to the current invention for the selection of clones based on product quality (part 1). Categorization for two analyzed antibody concentrations (50 ng/mL, 1.95 ng/mL) are shown. Displayed are 610 clones in total (FIGS. 11 and 12 together) producing diverse antibody quality.

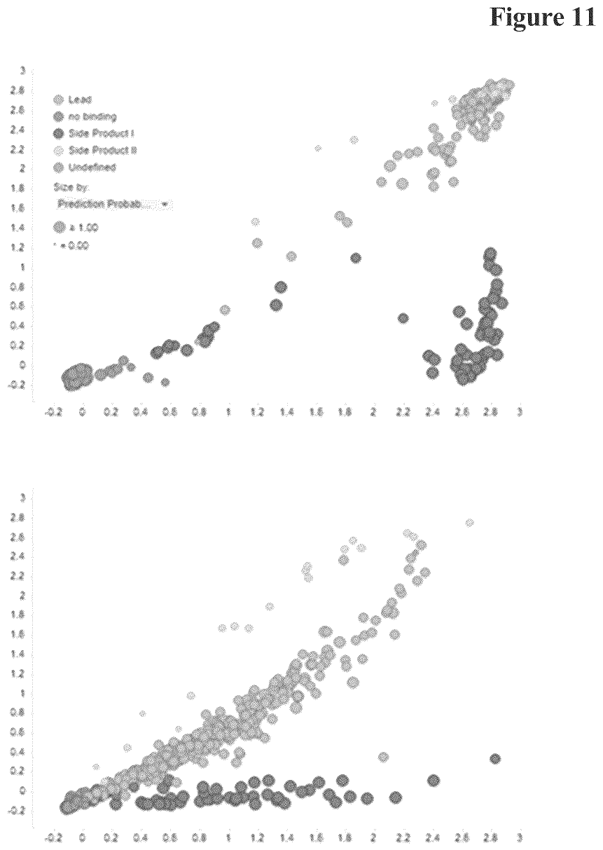

FIG. 12 Use of the method according to the current invention for the selection of clones based on product quality (part 2). Categorization for two analyzed antibody concentrations (9.9 ng/mL, 0.39 ng/mL) are shown. Displayed are 610 clones in total (FIGS. 11 and 12 together) producing diverse antibody quality.

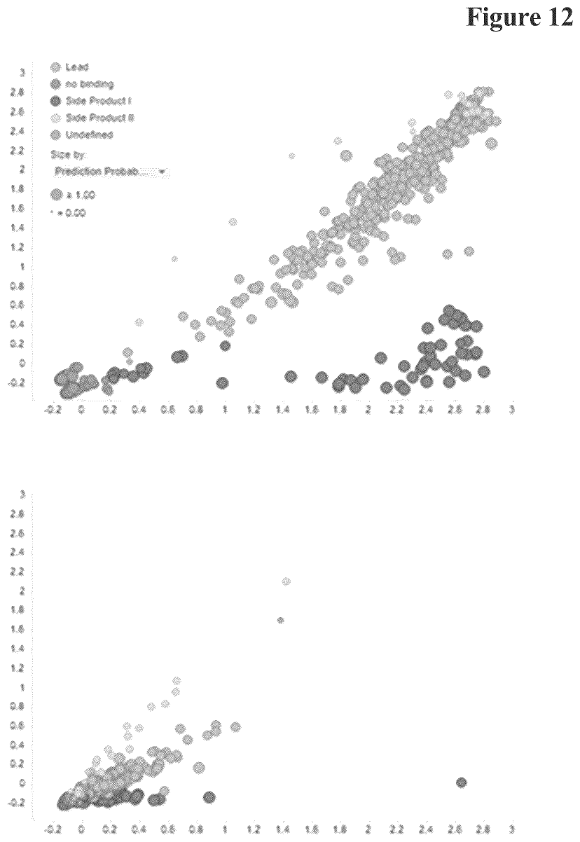

FIG. 13 Use of the method according to the current invention for the selection of clones based on product quality (part 3). Results for two analyzed antibody concentrations (50 ng/mL, 1.95 ng/mL) are shown. Displayed are 160 clones in total (FIGS. 13 and 14 together) selected with the method according to the current invention based on antibody quality.

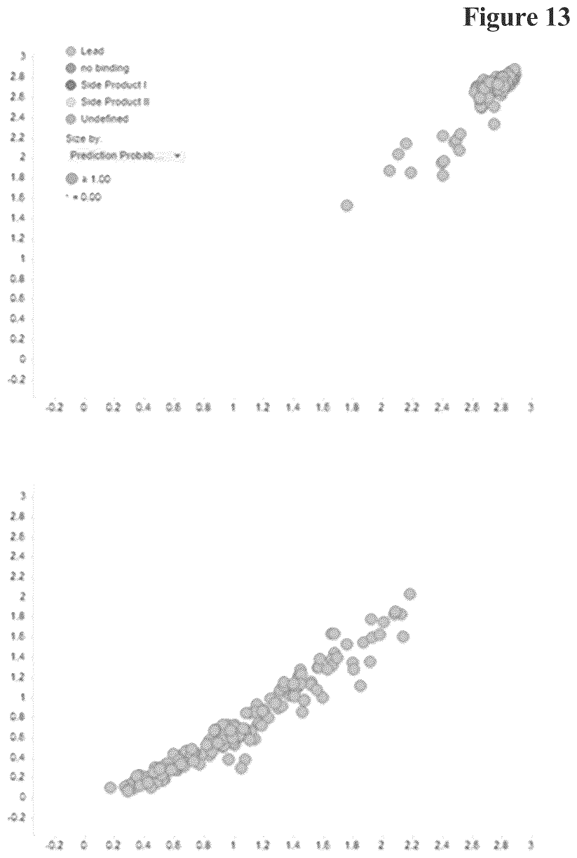

FIG. 14 Use of the method according to the current invention for the selection of clones based on product quality (part 4). Results for two analyzed antibody concentrations (9.9 ng/mL, 0.39 ng/mL) are shown. Displayed are 160 clones in total (FIGS. 13 and 14 together) selected with the method according to the current invention based on antibody quality.

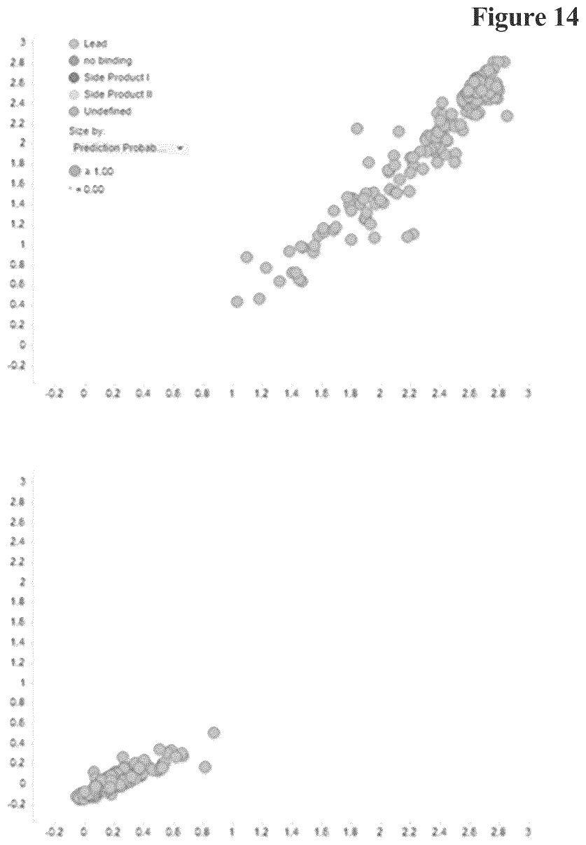

FIG. 15 Bispecific antibody formats and their common side-products.

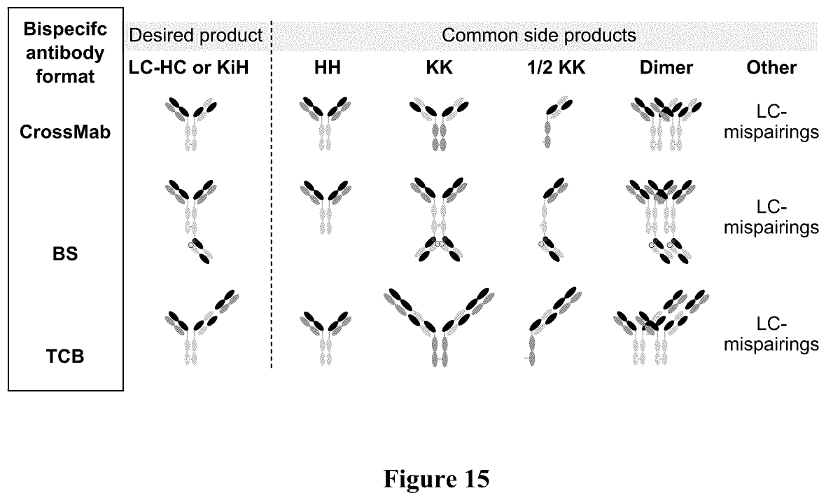

FIG. 16 Product titer based on clone.

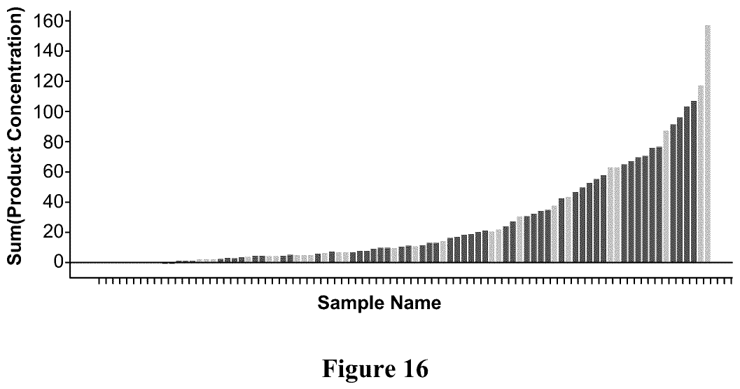

FIG. 17 Relation of SEC-determined monomer content (x-axis) and amount of main product determined by CE-SDS (y-axis) of clones of FIG. 16; clones deselected with the method according to the invention are shown in bold.

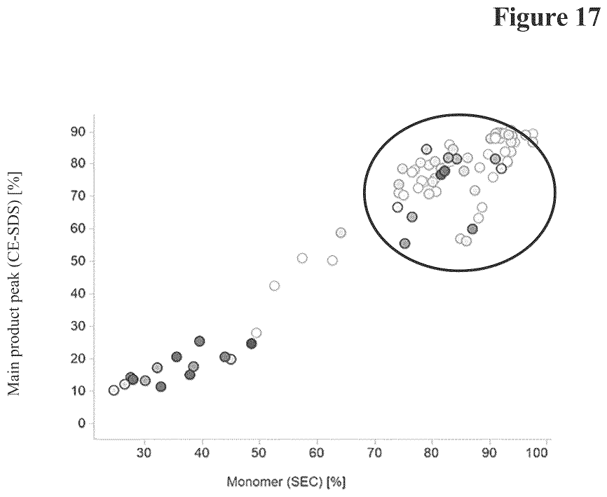

FIG. 18 Relation of SEC-determined monomer content (x-axis) and amount of main product determined by HIC (y-axis) of clones of FIG. 16; clones deselected with the method according to the invention are shown in bold.

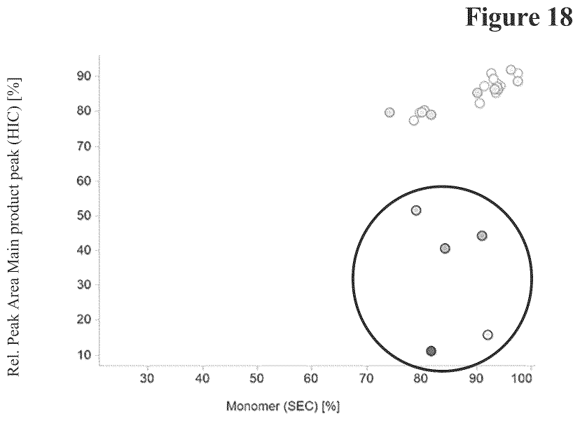

FIG. 19 Relation of SEC-determined monomer content (x-axis) and amount of main product determined by CE-SDS (y-axis) of clones of FIG. 16; clones selected with the method according to the invention are shown in bold.

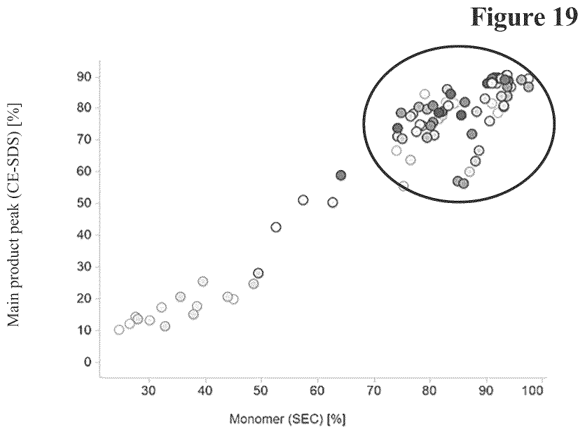

FIG. 20 Relation of SEC-determined monomer content (x-axis) and amount of main product determined by HIC (y-axis) of clones of FIG. 16; clones selected with the method according to the invention are shown in bold.

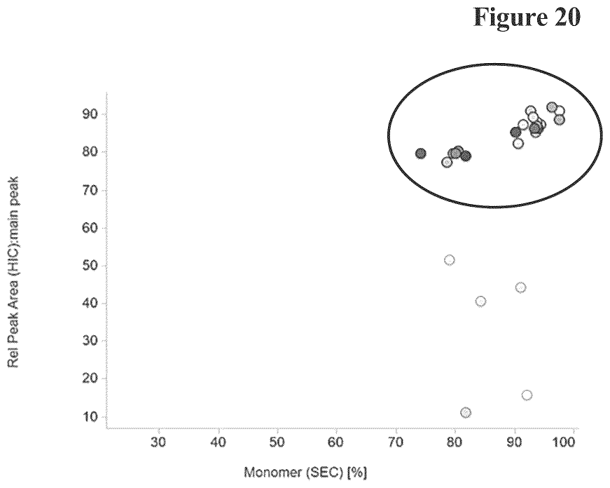

### EXAMPLES

### Example 1

General Techniques

1) Recombinant DNA Techniques

Standard methods were used to manipulate DNA as described in Sambrook et al., Molecular Cloning: A Laboratory Manual, Second Edition, Cold Spring Harbor Laboratory Press, Cold Spring Harbor, N.Y, (1989). The molecular biological reagents were used according to the manufacturer's instructions.

2) DNA Sequence Determination

DNA sequencing was performed at SequiServe GmbH (Vaterstetten, Germany) 3) DNA and protein sequence analysis and sequence data management The EMBOSS (European Molecular Biology Open Software Suite) software package and Invitrogen's Vector NTI version 11.5 were used for sequence creation, mapping, analysis, annotation and illustration.

4) Gene and Oligonucleotide Synthesis

Desired gene segments were prepared by chemical synthesis at Geneart GmbH (Regensburg, Germany). The synthesized gene fragments were cloned into an E. coli plasmid for propagation/amplification. The DNA sequences of subcloned gene fragments were verified by DNA sequencing. Alternatively, short synthetic DNA fragments were assembled by annealing chemically synthesized oligonucleotides or via PCR. The respective oligonucleotides were prepared by metabion GmbH (Planegg-Martinsried, Germany).

5) Reagents

All commercial chemicals, antibodies and kits were used as provided according to the manufacturer's protocol if not stated otherwise.

6) Cultivation of Host Cell Line CHO host cells were cultivated at 37° C. in a humidified incubator with 85% humidity and 5% CO₂. They were cultivated in a proprietary DMEM/F12-based medium containing selection agents. The cells were splitted every 3 or 4 days. For the cultivation 125 ml non-baffled Erlenmeyer shake-flasks were used. Cells were shaken at 150 rpm with a shaking amplitude of 5 cm. The cell count was determined with Cedex HiRes Cell Counter (Roche). Cells were kept in culture until they reached an age of 60 days.

7) Cloning

Cloning of nucleic acids has been done based on procedures known to a person skilled in the art or according to manufacturer's protocols.

Generally, cloning with R-sites depends on DNA sequences next to the gene of interest (GOI) that are equal to sequences lying in following fragments. Like that, assembly of fragments is possible by overlap of the equal sequences and subsequent sealing of nicks in the assembled DNA by a DNA ligase. After successful cloning of these preliminary vectors the gene of interest flanked by the R-sites is cut out via restriction digest by enzymes cutting directly next to the R-sites. For the assembly of all DNA fragments in one step, a 5′-exonuclease removes the 5′-end of the overlapping regions (R-sites). After that, annealing of the R-sites can take place and a DNA polymerase extends the 3′-end to fill the gaps in the sequence. Finally, the DNA ligase seals the nicks in between the nucleotides. Addition of an assembly master mix containing different enzymes like exonucleases, DNA polymerases and ligases, and subsequent incubation of the reaction mix at 50° C. leads to an assembly of the single fragments to one plasmid. After that, competent E. coli cells are transformed with the plasmid.

For some vectors, a cloning strategy via restriction enzymes was used. By selection of suitable restriction enzymes, the wanted gene of interest can be cut out and afterwards inserted into a different vector by ligation. Therefore, enzymes cutting in a multiple cloning site (MCS) are preferably used and chosen in a manner, so that a ligation of the fragments in the correct array can be conducted. If vector and fragment are previously cut with the same restriction enzyme, the sticky ends of fragment and vector fit together and can be ligated by a DNA ligase, subsequently. After ligation, competent E. coli cells are transformed with the newly generated plasmid.

For transformation, 10-beta competent E. coli cells were used according to the manufacturer's protocol. Only bacteria, which have successfully incorporated the plasmid, carrying the resistance gene against ampicillin, can grow on the respective plates. Single colonies were picked and cultured in LB-Amp medium for subsequent plasmid preparation.

### Bacterial Culture

Cultivation of E. coli was done in LB-medium, short for Luria Bertani, which was spiked with 1 ml/L 100 mg/ml ampicillin resulting in an ampicillin concentration of 0.1 mg/ml.

A 15 ml-tube (with a ventilated lid) was filled with 3.6 ml LB-Amp medium and equally inoculated with a bacterial colony. The toothpick was not removed but left in the tube during incubation. The tubes were incubated at 37° C., 200 rpm for 23 hours. The 15 ml tubes were taken out of the incubator and the 3.6 ml bacterial culture splitted into two 2 ml Eppendorf tubes. The tubes were centrifuged at 6,800×g in a tabletop microcentrifuge for 3 minutes at room temperature. After that, Mini-Prep was performed with the Qiagen QIAprep Spin Miniprep Kit according to the manufacturer's instructions. The plasmid DNA concentration was measured with Nanodrop.

### Ethanol Precipitation

The volume of the DNA solution was mixed with the 2.5-fold volume ethanol 100%. The mixture was incubated at −20° C. for 10 min. Then the DNA was centrifuged for 30 min. at 14,000 rpm, 4° C. The supernatant was carefully removed and the pellet washed with 70% ethanol. Again, the tube was centrifuged for 5 min. at 14,000 rpm, 4° C. The supernatant was carefully removed by pipetting and the pellet dried. When the ethanol was evaporated, an appropriate amount of endotoxin-free water was added. The DNA was given time to re-dissolve in the water overnight at 4° C. A small aliquot was taken and the DNA concentration was measured with a Nanodrop device.

### Example 2

### Plasmid Generation

Expression Cassette Composition

For the expression of an antibody chain, a transcription unit comprising the following functional elements was used:
the immediate early enhancer and promoter from the human cytomegalovirus including intron A,
a human heavy chain immunoglobulin 5′-untranslated region (5′UTR),
a murine immunoglobulin heavy chain signal sequence,
a nucleic acid encoding the respective antibody chain,
the bovine growth hormone polyadenylation sequence (BGH pA), and
optionally the human gastrin terminator (hGT).

Beside the expression unit/cassette including the desired gene to be expressed, the basic/standard mammalian expression plasmid contains
an origin of replication from the vector pUC18 which allows replication of this plasmid in E. coli, and
a beta-lactamase gene which confers ampicillin resistance in E. coli.

### Front- and Back-Vector Cloning

To construct two-plasmid antibody constructs, antibody HC and LC were cloned into a front vector backbone containing L3 and LoxFAS sequences, and a back vector containing LoxFAS and 2L sequences and a selectable marker. The Cre recombinase plasmid pOG231 (Wong, E. T., et al., Nucl. Acids Res. 33 (2005) e147; O'Gorman, S., et al., Proc. Natl. Acad. Sci. USA 94 (1997) 14602-14607) was used for all RMCE processes.

The cDNAs encoding the respective antibody chains were generated by gene synthesis (Geneart, Life Technologies Inc.). The gene synthesis and the backbone-vectors were digested with HindIII-HF and EcoRI-HF (NEB) at 37° C. for 1 h and separated by agarose gel electrophoresis. The DNA-fragment of the insert and backbone were cut out from the agarose gel and extracted by QIAquick Gel Extraction Kit (Qiagen). The purified insert and backbone fragment was ligated via the Rapid Ligation Kit (Roche) following the manufacturer's protocol with an Insert/Backbone ratio of 3:1. The ligation approach was then transformed in competent E. coli DH5α via heat shock for 30 sec. at 42° C. and incubated for 1 h at 37° C. before they were plated out on agar plates with ampicillin for selection. Plates were incubated at 37° C. overnight.

On the following day clones were picked and incubated overnight at 37° C. under shaking for the Preparation, which was performed with the EpMotion® 5075 (Eppendorf) or with the QIAprep Spin Mini-Prep Kit (Qiagen)/NucleoBond Xtra Maxi EF Kit (Macherey & Nagel), respectively. All constructs were sequenced to ensure the absence of any undesirable mutations (SequiServe GmbH).

In the second cloning step, the previously cloned vectors were digested with KpnI-HF/SalI-HF and SalI-HF/MfeI-HF with the same conditions as for the first cloning. The backbone vector was digested with KpnI-HF and MfeI-HF. Separation and extraction was performed as described above. Ligation of the purified insert and backbone was performed using T4 DNA Ligase (NEB) following the manufacturing protocol with an Insert/Insert/Backbone ratio of 1:1:1 overnight at 4° C. and inactivated at 65° C. for 10 min. The following cloning steps were performed as described above.

### Example 3

### Cultivation, Transfection, Selection and Single Cell Cloning

Host cells were propagated in disposable 125 ml vented shake flasks under standard humidified conditions (95% rH, 37° C., and 5% CO₂) at a constant agitation rate of 150 rpm in a proprietary DMEM/F12-based medium. Every 3-4 days the cells were seeded in chemically defined medium containing selection marker 1 and selection marker 2 in effective concentrations with a concentration of 3×10E5 cells/ml. Density and viability of the cultures were measured with a Cedex HiRes cell counter (F. Hoffmann-La Roche Ltd, Basel, Switzerland).

For stable transfection, equimolar amounts of front and back vector were mixed. 1 μg Cre expression plasmid was added to 5 μg of the mixture.

Two days prior to transfection host cells were seeded in fresh medium with a density of 4×10E5 cells/ml. Transfection was performed with the Nucleofector device using the Nucleofector Kit V (Lonza, Switzerland), according to the manufacturer's protocol. 3×10E7 cells were transfected with 30 μg plasmid. After transfection, the cells were seeded in 30 ml medium without selection agents.

On day 5 after seeding the cells were centrifuged and transferred to 80 mL chemically defined medium containing selection agent 1 and selection agent 2 at effective concentrations at 6×10E5 cells/ml for selection of recombinant cells. The cells were incubated at 37° C., 150 rpm. 5% CO2, and 85% humidity from this day on without splitting. Cell density and viability of the culture was monitored regularly. When the viability of the culture started to increase again, the concentrations of selection agents 1 and 2 were reduced to about half the amount used before.

Ten days after starting selection, the success of Cre mediated cassette exchange was checked by flow cytometry measuring the expression of intracellular marker and bispecific antibody bound to the cell surface. An APC antibody (allophycocyanin-labeled F(ab′)2 Fragment goat anti-human IgG) against human antibody light and heavy chain was used for FACS staining. Flow cytometry was performed with a BD FACS Canto II flow cytometer (BD, Heidelberg, Germany). Ten thousand events per sample were measured. Living cells were gated in a plot of forward scatter (FSC) against side scatter (SSC). The live cell gate was defined with non-transfected host cells and applied to all samples by employing the FlowJo 7.6.5 EN software (TreeStar, Olten, Switzerland). Fluorescence of the intracellular marker was quantified in the FITC channel (excitation at 488 nm, detection at 530 nm). Bispecific antibody was measured in the APC channel (excitation at 645 nm, detection at 660 nm). Parental CHO cells, i.e. those cells used for the generation of the host cell, were used as a negative control with regard to intracellular marker and bispecific antibody expression. Fourteen days after the selection had been started, the viability exceeded 90% and selection was considered as complete.

### Example 4

### FACS Screening

FACS analysis was performed to check the transfection efficiency. 4×10E5 cells of the transfected approaches were centrifuged (1200 rpm, 4 min.) and washed twice with 1 mL PBS. After the washing steps with PBS the pellet was resuspended in 400 μL PBS and transferred in FACS tubes (Falcon® Round-Bottom Tubes with cell strainer cap; Corning). The measurement was performed with a FACS Canto II and the data were analyzed by the software FlowJo.

### Example 5

### Single Cell Cloning

Resistant pools were subjected to single cell cloning by limiting dilution in 384-well plates at 0.6 cells/well. Prior to seeding, the pools were stained with 5-chloromethylfluorescein diacetate (CMFDA), a live-cell fluorescent dye. After seeding, the plates were centrifuged and images were acquired from each well in both fluorescence and brightfield mode. Fluorescence imaging allows for the detection of living cells and differentiation against cell-like artefacts as observed in the brightfield image. Approximately two weeks after seeding, confluence was determined by brightfield imaging. Colonies that had been identified as originating from a single cell were expanded to 96-well plates and analyzed by ELISA for titer and binding to target antigens.

### Example 6

### ELISA-Based Titer Determination

A mixture of 0.25 μg/mL biotinylated F(ab′)2 goat anti-human IgG (Fcy spec.; Jackson ImmunoResearch, #109-066-098) and the detection antibody POD-conjugated F(ab′)2 goat anti-human IgG (Fcy spec; Jackson ImmunoResearch, #109-036-098) in dilution buffer (=1×PBS (0.01 M KH₂PO₄, 0.1 M Na₂HPO₄, 1.37 M NaCl, 0.027 M KCl, pH 7.0), 0.5% BSA, 0.05% Tween 20 (Sigma Aldrich, CAS #9005-64-5)) was added to a streptavidin-coated microtiter plate (Nunc, 384 Well, #840010; 20 μL/well). The control antibody (a TCB) was diluted to a starting concentration of 0.2 μg/mL in dilution buffer and 12 dilutions (dilution factor 1:2) were transferred to the assay plate (10 μL/well). The cultivation supernatants were added to the assay plate (10 μL/well) and the plate was incubated for one hour at room temperature w/o agitation. The wells were washed six times with 90 μL/well wash buffer each (=1×PBS (0.01 M KH₂PO₄, 0.1 M Na₂HPO₄, 1.37 M NaCl, 0.027 M KCl, pH 7.0), 0.1% Tween 20 (Sigma Aldrich, CAS #9005-64-5)). Afterwards, 30 μL/well detection substrate TMB (SeraCare, KPL SureBlue Reserve TMB Microwell Peroxidase Substrate, Cat #5120-0087) was added and incubated for five minutes at room temperature w/o agitation. The absorbance measurement was done at a wavelength of 370 nm using a Powerwave HT reader (BioTeK). All ELISA signals for samples and control antibody (raw data) were blank corrected by subtraction of the signal measured for a well containing only dilution buffer (blank-corrected data). The blank corrected signals of the 12 concentrations of control antibody were used to calculate a standard curve (linear regression). The antibody titer was calculated using the linear regression equation of the control antibody.

### Example 7

### ELISA-Based Binding Assays

A pre-mix of 0.03 μg/mL biotinylated antigen 1-human Fc-region (huFc) conjugate or 0.25 μg/mL biotinylated antigen 2-human Fc-region conjugate and the detection antibody anti-human F(ab)2-HRP (Jackson ImmunoResearch, #109-036-006; 1:5000 i.A.) in dilution buffer (=1×PBS (0.01 M KH₂PO₄, 0.1 M Na₂HPO₄, 1.37 M NaCl, 0.027 M KCl, pH 7.0), 0.5% BSA, 0.05% Tween 20 (Sigma Aldrich, CAS #9005-64-5)) was added to a streptavidin-coated microtiter plate (Nunc; 384 Well, #840010; 20 μL/well). Supernatants and control antibody were diluted to a starting concentration of 0.05 μg/mL in dilution buffer, transferred to the wells (5 μL/well) and incubated for one hour at room temperature w/o agitation. The purified antibody main product (Knob-into-Hole heterodimer) and two antibody side-products (Hole-Hole and Knob-Knob, respectively, homodimers) were added in 14 dilutions with a dilution factor 1:1.5 each, while the samples were applied in 4 dilutions with a dilution factor 1:5.0625 each. The wells were washed six times with 90 μL/well wash buffer (=1×PBS (0.01 M KH₂PO₄, 0.1 M Na₂HPO₄, 1.37 M NaCl, 0.027 M KCl, pH 7.0), 0.1% Tween 20 (Sigma Aldrich, CAS #9005-64-5)). Afterwards, 30 μL/well detection substrate TMB (SeraCare, KPL SureBlue Reserve TMB Microwell Peroxidase Substrate, Cat #5120-0087) was added and incubated for five minutes at room temperature w/o agitation. The absorbance measurement was done at a wavelength of 370 nm using an EnVision reader (Perkin Elmer).

### Example 8

### Bridging ELISA AG12

Non-biotinylated antigen 2-huFc conjugate was directly coated in a concentration of 0.5 μg/mL in dilution buffer to a microtiter plate (Nunc, MaxiSorp, #464718) and incubated over night at 4° C. w/o agitation. After washing the plate (3× 90 μL/well with wash buffer each; 1×PBS (0.01 M KH₂PO₄, 0.1 M Na₂HPO₄, 1.37 M NaCl, 0.027 M KCl, pH 7.0), 0.1% Tween 20 (Sigma Aldrich, CAS #9005-64-5)) and blocking with 90 μL Blocking buffer (2% BSA in dilution buffer) for 30 min. and washing the plate again (3×90 μL/well with wash buffer each), the samples and control antibodies were added and incubated for one hour at room temperature. The purified main product (Knob-into-Hole heterodimer) and the two main side products (Hole-Hole and Knob-Knob, respectively, homodimers) were added in 14 dilutions with a dilution factor 1:1.5 each, while the samples were applied in 4 dilutions with a dilution factor 1:5.0625 each. After washing the plate (3×90 μL/well with wash buffer), 25 μL of a mixture of biotinylated antigen 1-huFc conjugate and Streptavidin POD-conjugate (Roche Diagnostics GmbH, Mannheim, Germany) was applied. The wells were washed six times with 90 μL/well wash buffer each. Afterwards, 30 μL/well detection substrate TMB (SeraCare, KPL SureBlue Reserve TMB Microwell Peroxidase Substrate, Cat #5120-0087) was added and incubated for five minutes at room temperature w/o agitation. The absorbance measurement was done at a wavelength of 370 nm using an EnVision reader (Perkin Elmer).

### Example 9

### Machine Learning

### Input Data

For each cell clone, the blank-corrected readouts form the ELISA assays for antibody titer, binding to antigen 1, binding to antigen 2 and simultaneous binding to antigen 1 and 2 were used as input data.

As a target variable (referred to as label in machine learning) the classification into five different product categories (Lead, Side Product I, Side Product II, No-Binding and Undefined) of cell clone supernatants by a human expert from historic projects was utilized.

Further manipulations of the data were performed (feature engineering) and entered into the model in addition to the input data.

### Feature Engineering

Eight different data processing variants have been used for building predictive models. Therefore, the blank-corrected readout from the ELISA assays was transformed in different ways as shown in the following Table.

| | number | |
|---|---|---|
| processing | standard | |
| variant no | concentrations | analyzed dataset includes |
| 1 | not applicable | titer, sample data |
| 2 | 4 | titer, sample data, standards in 4 concentrations |
| 3 | 4 | titer, sample data, 1-standard-normalized samples |
| 4 | 4 | titer, sample data, 3-standard-normalized samples |
| 5 | 4 | titer, sample data, ratios, 9 normalized ratios |
| 6 | 14 | titer, sample data, ratios, 9 normalized ratios |
| 7 | 14 | titer, sample data, 9 normalized ratios |
| 8 | 14 | titer, sample data, 4 normalized ratios |

| | |
|---|---|
| titer | total antibody titer, determined by ELISA using 8 dilutions of |
| | the sample |
| sample | supernatant of respective cell clone |
| sample data | absorbance corrected ELISA signals for 4 sample |
| | concentrations in 3 different ELISA assays (binding to AG1, |
| | binding to AG2, bridging of AG1 and AG2) |
| standards | purified (100%) antibody main product, side-product I and |
| | side-product II, respectively |
| standards in 4 | standards are evaluated using mean of 4 analyzed sample |
| concentrations | concentrations |
| standards in 14 | standards are evaluated using average of 14 analyzed sample |
| concentrations | concentrations |
| normalized | calculated ratio to standard |
| 1-standard- | sample data normalized to main product standard |
| normalized | |
| samples | |
| 3-standard | sample data normalized to main product, side-product I and |
| normalized | side-product II standards |
| samples | |
| ratios | ratio of sample data AG2 to AG1, AG12 to AG1, and AG12 |
| | to AG2 |
| 3-standard- | ratios normalized to main product, side-product I and side- |
| normalized | product II standards |
| ratios | |
| 9 normalized | AG2:AG1, AG12:AG1 and AG12:AG2 ratios each |
| ratios | normalized to main product, side-product I and side-product II |
| | standards |
| 4 normalized | AG2: AG1 ratio normalized to main product standard, |
| ratios | AG12:AG1 ratio normalized to main product standard, |
| | AG2: AG1 ratio normalized to side-product I standard, |
| | AG12:AG1 normalized to side-product II standard |

In detail, the processing variants were as follows:

Processing variant no. 1: only the binding and bridging ELISA assay data for the supernatants (absorbance corrected ELISA signals of supernatants of candidate clones) and the determined total antibody titer were used; this is the baseline experiment; the utilized data were part of all consecutive experiments.

Processing variant nos. 2-8: assay readouts for the three standards Lead (100% purified main product), Side Product I (100% purified side product I, hole-hole homodimer) and Side Product 11 (100% purified side product II, knob-knob homodimer) were added to the dataset (variant 2) or used in modified form (variants 3-8); each standard was determined in 14 different concentrations (standard curve); for variants 2-5, four out of these concentrations in the linear assay range were selected for the calculation.

For variants 6-8 median values of all 14 analyzed concentrations were used, hence not requiring a selection step.

Variant nos. 3-8 contained data, which were normalized against data from one or all three standards. Variant 3 utilized sample values normalized against the Lead standard, whereas variant 4 worked with values normalized against all three standards.

Variant nos. 5-8 contained ratios instead of simple values, which were utilized as values normalized against all three standards. Out of the 9 possible normalized ratios, variant 7 contained all 9, whereas variant 8 utilized just 4 selected ones.

### Training

For training, three projects were pooled and the fourth project held back for final model evaluation (unseen test dataset). The training data was then used for model building and hyperparameter optimization. The driverless AI tool form H2O.ai was used to automatically generate machine-learning models. Each processing variant (see Table above) was used to generate a dataset, on which the training and evaluation was performed.

### Testing

Only the final model (with the best hyperparameter set) was then scored by applying the cross-entropy loss function to the unseen test dataset (project four).

### REFERENCES

Scikit-learn: Machine Learning in Python, Pedregosa et al., JMLR 12, pp. 2825-2830, 2011.

1. A method for selecting one or more recombinant cell clones from a multitude of recombinant cell clones all expressing the same recombinant bispecific antibody binding to a first antigen and a second antigen comprising the following steps:
cultivating each single deposited recombinant cell clone of the multitude of recombinant cell clones to produce a cultivation supernatant;
determining
i) the total antibody concentration in the supernatant;
ii) the binding signal for each supernatant for binding to the first antigen of the bispecific antibody in an immunoassay for 4 different concentrations of the supernatant;
iii) the binding signal for each supernatant for binding to the second antigen of the bispecific antibody in an immunoassay for the 4 same concentrations of the supernatant as in ii);
iv) the binding signal for each supernatant for simultaneous binding to the first and the second antigen of the bispecific antibody in an immunoassay for the 4 same concentrations of the supernatant as in ii);
v) the binding signal for the isolated bispecific antibody for binding to the first, second and both antigens with the same immunoassay as in ii), iii) and iv) each for 14 different concentrations of the bispecific antibody;
vi) the binding signal for a first isolated bispecific antibody-related side product that is also expressed by said recombinant cell clones for binding to the first, second and both antigens with the same immunoassay as in ii), iii) and iv) each for the 14 same concentrations as in v) of the first bispecific antibody-related side product;
vii) the binding signal for a second isolated bispecific antibody-related side product that is also expressed by said recombinant cell clones for binding to the first, second and both antigens with the same immunoassay as in ii), iii) and iv) each for the 14 same concentrations as in v) of the second bispecific antibody-related side product;
ranking the recombinant cell clones of the multitude of recombinant cell clones based on the following values
the concentration of i),
the binding signal of ii), iii), and iv),
the median of the individual ratios of the binding signal for each of the concentrations obtained in iii) to the respective binding signals for the same concentration obtained in ii) divided by the median of the individual ratios of the binding signal for each of the concentrations obtained in v) using the immunoassay of iii) to the respective binding signal for the same concentration obtained in v) using the immunoassay of ii),
the median of the individual ratios of the binding signal for each of the concentrations obtained in iv) to the respective binding signals for the same concentration obtained in ii) divided by the median of the individual ratios of the binding signal for each of the concentrations obtained in v) using the immunoassay of iv) to the respective binding signal for the same concentration obtained in v) using the immunoassay of ii),
the median of the individual ratios of the binding signal for each of the concentrations obtained in iii) to the respective binding signals for the same concentration obtained in ii) divided by the median of the individual ratios of the binding signal for each of the concentrations obtained in vi) using the immunoassay of iii) to the respective binding signal for the same concentration obtained in vi) using the immunoassay of ii), and
the median of the individual ratios of the binding signal for each of the concentrations obtained in iv) to the respective binding signals for the same concentration obtained in ii) divided by the median of the individual ratios of the binding signal for each of the concentrations obtained in vii) using the immunoassay of iv) to the respective binding signal for the same concentration obtained in vii) using the immunoassay of ii),
whereby the ranking is by the respective difference of the above values for each individual cell clone to the values obtained for the isolated bispecific antibody,
whereby the recombinant cell clones with the smallest overall difference are selected.

2. The method according to claim 1, wherein the difference is determined using an in silico method.

3. The method according to claim 1, wherein the difference is determined using machine learning.

4. The method according to claim 1, wherein the difference is determined using an extreme gradient boosting model.

5. The method according to claim 1, wherein the selection is based on the smallest difference with respect to the concentration, the binding signals and the bispecific antibody divided ratios in combination with the biggest difference with respect to the side product divided ratios.

6. The method according to claim 1, wherein the one or more selected cell clones express said recombinant bispecific antibody with lower percentage of antibody-related side products as the average of the multitude of recombinant cell clones.

7. The method according to claim 1, wherein the bispecific antibody comprises a heterodimeric Fc-region, wherein one Fc-region polypeptide comprises the knob-mutation and the respective other Fc-region polypeptide comprises the hole-mutations; the first bispecific antibody-related side product is the hole-hole homodimer of said bispecific antibody; the second bispecific antibody-related side product is the knob-knob homodimer of said bispecific antibody.

8. The method according to claim 1, wherein the immunoassay is an enzyme-linked immunosorbent assay.

9. The method according to claim 1, wherein the cell is a CHO cell.

10. Use of
i) the total antibody concentration determined in a single recombinant cell clone cultivation supernatant, wherein the recombinant cell clone has been transfected with nucleic acids encoding a bispecific antibody,
ii) the binding signals for isolated binding of the supernatant to the first and second antigen of the bispecific antibody,
iii) the binding signals for simultaneous binding of the supernatant to the first and second antigen of the bispecific antibody
for identifying recombinant cell clones expressing said bispecific antibody with low bispecific antibody-related side products.

11. The use according to claim 10, wherein further
iv) the binding signals for isolated and simultaneous binding of the isolated bispecific antibody) to its first and second antigen,
v) the binding signals for isolated and simultaneous binding of a first isolated bispecific antibody-related side product to the first and second antigen of the bispecific antibody,
vi) the binding signals for isolated and simultaneous binding of a second isolated bispecific antibody-related side product to the first and second antigen of the bispecific antibody
are used for identifying recombinant cell clones expressing said bispecific antibody with low bispecific antibody-related side products.

12. The use according to claim 1, wherein further
the median of the individual ratios of the binding signal for each of the concentrations obtained in iii) to the respective binding signals for the same concentration obtained in ii) divided by the median of the individual ratios of the binding signal for each of the concentrations obtained in v) using the immunoassay of iii) to the respective binding signal for the same concentration obtained in v) using the immunoassay of ii),
the median of the individual ratios of the binding signal for each of the concentrations obtained in iv) to the respective binding signals for the same concentration obtained in ii) divided by the median of the individual ratios of the binding signal for each of the concentrations obtained in v) using the immunoassay of iv) to the respective binding signal for the same concentration obtained in v) using the immunoassay of ii),
the median of the individual ratios of the binding signal for each of the concentrations obtained in iii) to the respective binding signals for the same concentration obtained in ii) divided by the median of the individual ratios of the binding signal for each of the concentrations obtained in vi) using the immunoassay of iii) to the respective binding signal for the same concentration obtained in vi) using the immunoassay of ii), and
the median of the individual ratios of the binding signal for each of the concentrations obtained in iv) to the respective binding signals for the same concentration obtained in ii) divided by the median of the individual ratios of the binding signal for each of the concentrations obtained in vii) using the immunoassay of iv) to the respective binding signal for the same concentration obtained in vii) using the immunoassay of ii),
are used for identifying recombinant cell clones expressing said bispecific antibody with low bispecific antibody-related side products,
wherein
iv) is the binding signals for isolated and simultaneous binding of the bispecific antibody to its first and second antigen,
v) is the binding signals for isolated and simultaneous binding of a first isolated bispecific antibody-related side product to the first and second antigen of the bispecific antibody,
vi) is the binding signals for isolated and simultaneous binding of a second isolated bispecific antibody-related side product to the first and second antigen of the bispecific antibody.

13. A computer program including computer-executable instructions for performing the method according to claim 1 when the program is executed on a computer or computer network.

14. A computer program product having program code means in order to perform the method according to claim 1 when the program is executed on a computer or computer network.

15. A data carrier having a data structure stored thereon, which, after loading into a computer or computer network, such as into a working memory or main memory of the computer or computer network, may execute the method according to any claim 1.
